(19) United States
(12) Patent Application Publication
(10) Pub. No.: US 2024/0390403 A1
(43) Pub. Date: Nov. 28, 2024

(54) COMPOSITION AND METHODS FOR TREATING AN ANEURYSM

(71) Applicant: Nectero Medical, Inc., Tempe, AZ (US)

(72) Inventor: Alexander Schwarz, Newton, MA (US)

(21) Appl. No.: 18/674,433

(22) Filed: May 24, 2024

Related U.S. Application Data
(60) Provisional application No. 63/643,692, filed on May 7, 2024.
(60) Provisional application No. 63/504,375, filed on May 25, 2023.

(51) Int. Cl. A61K 31/7024; A61K 9/00; A61K 31/09; A61K 31/7016; A61K 49/04; A61P 9/00; A61P 9/14

(52) U.S. Cl. CPC A61K 31/7024; A61K 9/0019; A61K 31/09; A61K 31/7016; A61K 49/0438; A61P 9/00; A61P 9/14

(57) ABSTRACT

Disclosed herein are pollygalloyl compounds of Formula (I), (II), (III), (IV), (V), (VI), (VII), (VIII), (IX), (X), or (XI), including pharmaceutically acceptable salts thereof, and methods and uses thereof. Compounds disclosed herein may be provided in a kit, including a solubilizer for dissolving the compound or composition as described herein and a saline solution. Also disclosed herein are methods for delivery of a therapeutic solution to a blood vessel.

### CROSS-REFERENCE TO RELATED APPLICATIONS

The present application claims the benefit of priority to U.S. Provisional Application No. 63/643,692, filed May 7, 2024, and U.S. Provisional Application No. 63/504,375, filed May 25, 2023. The entirety of each of the foregoing applications is hereby incorporated by reference herein.

### BACKGROUND

One of the most common results of the degradation of vasculature is aneurysm. By definition, the term “aneurysm” is simply an abnormal widening or ballooning at the wall of a blood vessel. Aneurysms are degenerative diseases characterized by destruction of arterial architecture and subsequent dilatation of the blood vessel that may eventually lead to fatal ruptures. Some common locations for aneurysms include the abdominal aorta (abdominal aortic aneurysm, AAA), thoracic aorta, and brain arteries. In addition, peripheral aneurysms of the leg, namely the iliac, popliteal and femoral arteries are prevalent locations of this vascular pathology.

Almost 10% of Americans over the age of 65 years have some degree of abdominal or thoracic aortic enlargement. It is estimated that 15,000 patients die each year from complications of aortic aneurysms (e.g., rupture, dissection); and abdominal aortic aneurysm (AAA) rupture is the 13th leading cause of death in the United States. Therefore, treatments and therapies are needed before aneurysm rupture in a patient.

### SUMMARY OF THE DISCLOSURE

Aspects of the disclosure relate to a composition comprising a compound Formula (I), (II), (III) (IV), (V), (VI), (VII), (VIII), (IX), (X), or (XI), a water-soluble solubilizer, and a buffer. In some embodiments, the composition further comprises three or more galloyl esters. In some embodiments, the water-soluble solubilizer is a contrast agent. In some embodiments, the contrast agent is a radiocontrast material. In some embodiments, the contrast agent is a radiocontrast material is a non-ionic radio-opaque contrast agent. In some embodiments, the non-ionic radio-opaque contrast agent is selected from the group consisting of iopamidol, iohexol, iodixanol, or a combination thereof. In some embodiments, the water-soluble solubilizer is from about 10 to about 30% (w/v) of the composition. In some embodiments, a buffer is selected from the group consisting of lactic acid/lactate, citric acid/citrate, carbonic acid/carbonate, phosphate buffer system, boronic acid/borate, acetic acid/acetate, or combinations thereof. In some embodiments, the composition is at a pH between 2 to 6. In some embodiments, the composition is at a pH between 3 to 5. In some embodiments, the composition further comprising an osmolality agent. In some embodiments, the osmolality agent is selected from potassium chloride, calcium chloride, sodium lactate, sodium chloride, dextrose, mannitol, sucrose, trehalose, propylene glycol, glycerin, or combinations thereof. In some embodiments, the composition further comprises N-hydroxyethylaminodiacetic acid, hydroxyethylenediaminetetraacetic acid, nitrilotriacetic acid (NTA), ethylenediaminetetraacetic acid (EDTA), disodium EDTA, N-hydroxyethyl-ethylenediaminetriacetic acid (HEDTA), disodium ethylenediaminetetraacetate (Na₂H₂EDTA), tetrasodium ethylenediaminetetraacetate (Na₄EDTA), and calcium disodium ethylenediaminetetraacetate (CaNa₂EDTA), diethylenetriaminepentaacetic acid (DTPA), and alanine-N,N-diacetic acid, and combinations thereof.

Aspects of the disclosure relate to methods of reducing or inhibiting growth of an aneurysm in a subject having an aneurysm. In some embodiments, the method comprises administering the composition described herein to the aneurysm. In some embodiments, the aneurysm is selected from an aortic aneurysm, a peripheral aneurysm, or a neuro aneurysm. In some embodiments, the aortic aneurysm is greater than 3 cm in diameter. In some embodiments, the composition is administered by local delivery. In some embodiments, the local delivery is intraluminal delivery. In some embodiments, the local delivery comprises a stent coated by the composition as described herein. In some embodiments, the local delivery comprises a balloon coated by the composition accordingly as described herein. In some embodiments, the local delivery comprises a weeping balloon by the composition as described herein. In some embodiments, the local delivery comprises a catheter device by the composition as described herein. In some embodiments, a site of the administration is accessed via a femoral artery of the subject. In some embodiments, the method comprises administering a contrast agent prior to administering the composition to the subject. In some embodiments, a site of the administration is accessed via an aortic artery. In some embodiments, the aneurysm is an abdominal aortic aneurysm. In some embodiments, the catheter device is about 20 cm to about 50 cm in length. In some embodiments, the catheter device is introduced through an access sheath. In some embodiments, the catheter device further includes at least one guidewire. In some embodiments, the at least one guidewire is received by a first central lumen.

### BRIEF DESCRIPTION OF THE DRAWINGS

The features and advantages of the compositions and methods described herein will become apparent from the following description, taken in conjunction with the accompanying drawings. These drawings depict certain aspects of the compositions and methods described in the present application, and thus, are not to be considered limiting. In the drawings, similar reference numbers or symbols typically identify similar components, unless context dictates otherwise. The drawings may not be drawn to scale.

FIG. 1 illustrates a schematic of nomenclature of sites (left) and definition of open angle θ and tangent angles at tips a and a′ (right).

FIG. 2 illustrates a bar graph describing opening angels of various treatment groups. Data represents Means+/−SEM degrees for n=6 for group.

FIG. 3 illustrates a table representing a statistical comparison among analogs.

FIG. 4 illustrates macro photographs of samples in 4 groups.

FIG. 5 illustrates opening angles of various treatment groups. Data represents Means+/−SEM degrees for n=9 for group.

FIG. 6 illustrates a table representing a statistical comparison among analogs.

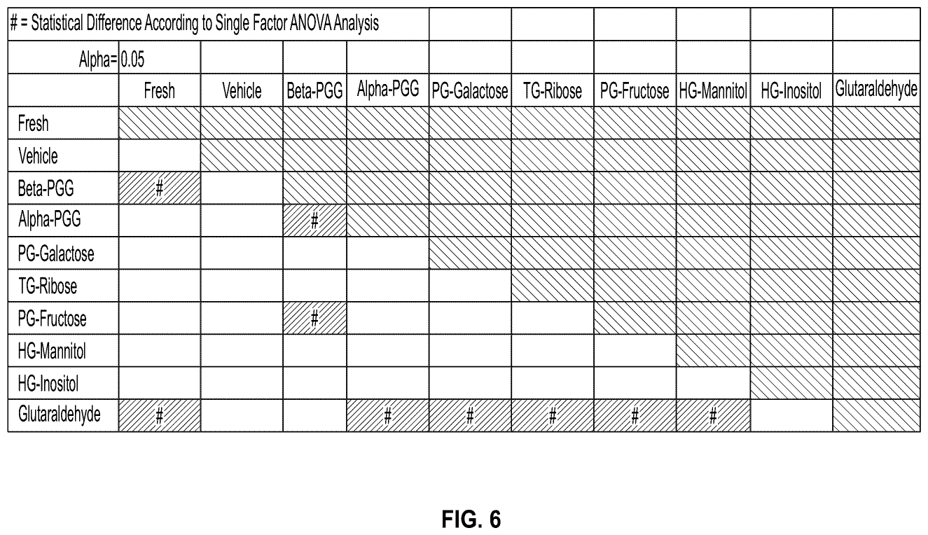

### DETAILED DESCRIPTION

Disclosed herein are compositions and methods useful for the prevention, treatment, and/or amelioration of various diseases, disorders, or conditions. Some embodiments pertain to compositions and methods for treating elastin depleted tissues. Also disclosed herein are compositions and methods of purifying and delivering a compound of Formula (I). (II). (III). (IV). (V), (VI). (VII), (VIII), (IX), (X), or (XI), disclosed elsewhere herein. Further disclosed herein, are devices for delivery of a compound of Formula (I), (II), (III). (IV). (V), (VI), (VII), (VIII), (IX), (X), or (XI), and/or in combination with another therapeutic agent to a blood vessel or other body lumen.

The section headings used herein are for organizational purposes only and are not to be construed as limiting the subject matter described. Features disclosed under one heading (such as a composition) can be used in combination with features disclosed under a different heading (a method of treating). Unless defined otherwise, all technical and scientific terms used herein have the same meaning as is commonly understood by one of ordinary skill in the art. It should be noted that the use of particular terminology when describing certain features or aspects of the disclosure should not be taken to imply that the terminology is being re-defined herein to be restricted to include any specific characteristics of the features or aspects of the disclosure with which that terminology is associated.

While the disclosure has been illustrated and described in detail in the foregoing description, such description is to be considered illustrative or exemplary and not restrictive. The disclosure is not limited to the disclosed embodiments. Variations to the disclosed embodiments can be understood and effected by those skilled in the art in practicing the claimed disclosure, from a study of the disclosure and the appended claims.

### Definitions

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including, “containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ preferred, ‘desired,’ or ‘desirable,’ and words of similar meaning should not be understood as implying that certain features are critical, essential, or even important to the structure or function of the disclosure, but instead as merely intended to highlight alternative or additional features that may or may not be utilized in a particular embodiment of the disclosure. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

As used in the specification and the appended claims, the singular forms “a,” “an” and “the” include plural referents unless the context clearly dictates otherwise. The use of “or” or “and” means “and/or” unless stated otherwise. Furthermore, use of the term “including” as well as other forms, such as “include”, “includes,” and “included,” is not limiting. The mere fact that certain measures are recited in mutually different dependent claims does not indicate that a combination of these measures cannot be used to advantage.

As used herein, the term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value. Where particular values are described in the application and claims, unless otherwise stated the term “about” meaning within an acceptable error range for the particular value should be assumed.

As used herein, a “carrier” refers to a compound that facilitates the delivery of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, the terms “effective amount” and “therapeutically effective amount” are broad terms, and are to be given their ordinary and customary meaning to a person of ordinary skill in the art (and are not to be limited to a special or customized meaning), and refer without limitation to a sufficient amount of an agent or a compound being administered which will relieve to some extent one or more of the symptoms of the disease and/or condition being treated. In some embodiments, the result can be reduction and/or alleviation of the signs, symptoms, or causes of a disease, or any other desired alteration of a biological system. For example, an “effective amount” for therapeutic uses is an amount of the composition comprising a compound as disclosed herein required to provide a clinically significant decrease in disease symptoms. An appropriate “effective” amount in any individual case may be determined using techniques, such as a dose escalation study. Where a drug has been approved by the U.S. Food and Drug Administration (FDA) or a counterpart foreign medicines agency, a “therapeutically effective amount” optionally refers to the dosage approved by the FDA or its counterpart foreign agency for treatment of the identified disease or condition.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

As used herein, the term “pharmaceutical composition” refers to a mixture of one or more compounds disclosed herein with other chemical components, such as diluents or carriers. The pharmaceutical composition facilitates administration of the compound to an organism. Pharmaceutical compositions will generally be tailored to the specific intended route of administration.

As used herein, the terms “treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

As used herein, the term “weight percent,” when referring to a component, is the weight of the component divided by the weight of the composition that includes the component, multiplied by 100%. For example, the weight percent of component A when 5 grams of component A is added to 95 grams of component B is 5% (e.g., 5 g A/(5 g A+95 g B)×100%).

### Compositions

Certain aspects of the disclosure relate to a composition including one or more polygalloyl compound. In some embodiments, the one or more polygalloyl compound is one or more monosaccharide represented by the formula (CH₂O)ₙ, where n may be 3, 4, 5, or 6 with each hydroxyl group can optionally be substituted with a galloyl group. In some embodiments, each hydroxyl group of the monosaccharide is substituted with a galloyl group. In some embodiments, the monosaccharide is in the keto form. In some embodiments, the monosaccharide is in aldo form. In some embodiments, the monosaccharide is a D-form monosaccharide. In some embodiments, the one or more polygalloyl compound is a disaccharide. In some embodiments, the disaccharide is represented by the formula (C₂ₙH₂ₙ₊₂O₂ₙ₋₁), where n is 3, 4, 5, or 6 with each hydroxyl group can optionally be substituted with a galloyl group. In some embodiments, the one or more polygalloyl compound is not pentagalloyl β-glucose. In some embodiments, the one or more polygalloyl compound may include, but is not limited to, polygalloyl mannose, polygalloyl galactose, polygalloyl xylose, polygalloyl ribose, polygalloyl arabinose, polygalloyl fructose, polygalloyl inositol, polygalloyl allose, polygalloyl talose, polygalloyl idose, polygalloyl gulose, polygalloyl erthrose, polygalloyl threose, polygalloyl sucrose, polygalloyl lactose, polygalloyl maltose, polygalloyl maltose, polygalloyl isomaltose, polygalloyl kojibiose, polygalloyl nigerose, polygalloyl laminaribiose, polygalloyl mannobiose, polygalloyl chitobiose, polygalloyl fucosyllactose, polygalloyl galactobiose, polygalloyl rutinose, polygalloyl neotrehalose, polygalloyl leucrose, polygalloyl trehalose, polygalloyl cellobiose, or a combination thereof.

Some embodiments provide a composition including a polygalloyl compound represented by a compound of Formula (I):

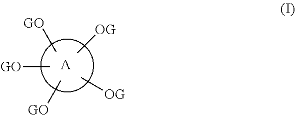

wherein
Ring A is a monosaccharide, wherein each —OH of the monosaccharide are substituted with OG,
G is galloyl group,
a water-soluble solubilizer, and
a buffer.

In some embodiments, Ring A is aldohexose. In some embodiments, the aldohexose is D-form. In some embodiments, the aldohexose is L-form. In some embodiments, the aldohexose is a combination of D- and L-forms. In some embodiments, the aldohexose is selected from the group consisting of allose, altrose, mannose, gulose, idose, galactose, glucose, talose, or a combination thereof. In some embodiments, Ring A is a ketohexose. In some embodiments, the ketohexose is D-form. In some embodiments, the ketohexose is L-form. In some embodiments, the ketohexose is a combination of D- and L-forms. In some embodiments, the ketohexose is selected from the group consisting of psicose, fructose, sorbose, tagatose. In some embodiments, Ring A is an aldopentose. In some embodiments, the aldopentose is D-form. In some embodiments, the aldopentose is L-form. In some embodiments, the aldopentose is a combination of D- and L-forms. In some embodiments, the aldopentose is selected from the group consisting of arabinose, lyxose, ribose, xylose. In some embodiments, the monosaccharide represented by the formula (CH₂O) n, where n may be 5 or 6 of Formula (I) are in alpha form. In some embodiments, the monosaccharide represented by the formula (CH₂O) n, where n may be 4, 5 or 6 of Formula (I) are in beta form. In some embodiments, the monosaccharide represented by the formula (CH₂O) n, where n may be 4, 5 or 6 of Formula (I) are in a combination of alpha and beta form. In some embodiments, the compound of Formula (I) is not pentagalloyl β-glucose. In some embodiments, the compound of Formula (I) is pentagalloyl alpha-glucose. In some embodiments, the compound of Formula (I) is pentagalloyl galactose. In some embodiments, the compound of Formula (I) is pentagalloyl maltose. In some embodiments, the compound of Formula (I) is pentagalloyl mannose. In some embodiments, the compound of Formula (I) is pentagalloyl ribose. In some embodiments, the compound of Formula (I) is pentagalloyl-fructose. In some embodiments, the compound of Formula (I) is polygalloyl inositol.

Some embodiments provide a composition including a one more polygalloyl compound represented by a compound of Formula (II):

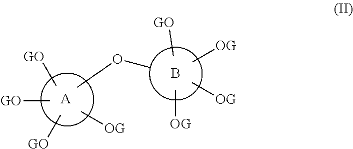

including pharmaceutically acceptable salts thereof, wherein
Ring A and B are each independently selected from a monosaccharide, wherein, each —OH of the carbohydrate are substituted with OG,
G is galloyl group,
a water-soluble solubilizer, and
a buffer.

In some embodiments, Ring A is aldohexose. In some embodiments, Ring B is aldohexose. In some embodiments, both Ring A and Ring B are aldohexoses. In some embodiments, both Ring A and Ring B are the same aldohexose. In some embodiments, Ring A and Ring B are different aldohexoses. In some embodiments, the aldohexose is D-form. In some embodiments, the aldohexose is L-form. In some embodiments, the aldohexose is a combination of D- and L-forms. In some embodiments, the aldohexose is selected from the group consisting of allose, altrose, mannose, gulose, idose, galactose, glucose, talose, or a combination thereof. In some embodiments, Ring A is ketohexose. In some embodiments, Ring B is ketohexose. In some embodiments, both Ring A and Ring B are ketohexose. In some embodiments, both Ring A and Ring B are the same ketohexose. In some embodiments, Ring A and Ring B are different ketohexose. In some embodiments, the ketohexose is D-form. In some embodiments, the ketohexose is L-form. In some embodiments, the ketohexose is a combination of D- and L-forms. In some embodiments, the ketohexose is selected from the group consisting of psicose, fructose, sorbose, tagatose. In some embodiments, Ring A is aldopentose. In some embodiments, Ring B is aldopentose. In some embodiments, both Ring A and Ring B are aldopentose. In some embodiments, both Ring A and Ring B are the same aldopentose. In some embodiments, Ring A and Ring B are different aldopentose. In some embodiments, the aldopentose is D-form. In some embodiments, the aldopentose is L-form. In some embodiments, the aldopentose is a combination of D- and L-forms. In some embodiments, the aldopentose is selected from the group consisting of arabinose, lyxose, ribose, xylose.

In some embodiments, the 5-membered ring carbohydrate or a 6-carbon ring carbohydrate of Formula (II) are in alpha form. In some embodiments, the 5-membered ring carbohydrate or a 6-carbon ring carbohydrate of Formula (II) are in beta form. In some embodiments, the composition includes 5-membered ring carbohydrate or a 6-carbon ring carbohydrate of Formula (II) in a combination of alpha and beta forms. In some embodiments, the 5-membered ring carbohydrate or a 6-carbon ring carbohydrate of Ring A of Formula (II) are in alpha form. In some embodiments, the 5-membered ring carbohydrate or a 6-carbon ring carbohydrate of Ring A of Formula (II) are in beta form. In some embodiments, the composition includes 5-membered ring carbohydrate or a 6-carbon ring carbohydrate of Ring A of Formula (II) in a combination of alpha and beta form. In some embodiments, the 5-membered ring carbohydrate or a 6-carbon ring carbohydrate of Ring B of Formula (II) are in alpha form. In some embodiments, the 5-membered ring carbohydrate or a 6-carbon ring carbohydrate of Ring B of Formula (II) are in beta form. In some embodiments, composition includes 5-membered ring carbohydrate or a 6-carbon ring carbohydrate of Ring B of Formula (II) in a combination of alpha and beta form. In some embodiments, the compound of Formula (II) is polygalloyl-sucrose.

Some embodiments relate to a composition including a compound of Formula (III):

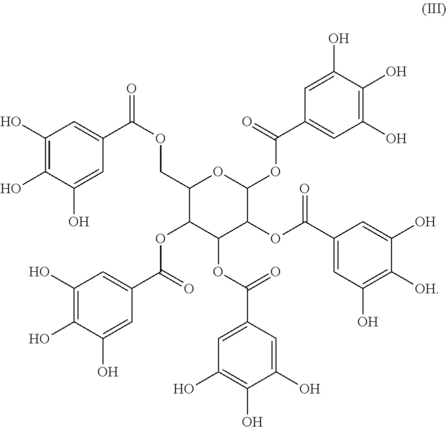 [SMILES: O=C(OCC1OC(OC(=O)c2cc(O)c(O)c(O)c2)C(OC(=O)c2cc(O)c(O)c(O)c2)C(OC(=O)c2cc(O)c(O)c(O)c2)C1OC(=O)c1cc(O)c(O)c(O)c1)c1cc(O)c(O)c(O)c1]

In some embodiments, Formula (III) C1 carbon can be in either alpha, beta, or a mixture of the two forms. In some embodiments, Formula (III) is either D- or L-form. In some embodiments, Formula (III) is selected from the group consisting of allose, altrose, mannose, gulose, idose, galactose, glucose, or talose. In some embodiments, the compound of Formula (III) is pentagalloyl α-glucose. In some embodiments, the compound of Formula (III) is not pentagalloyl β-glucose. In some embodiments, the compound of Formula (III) is in an alpha form. In some embodiments, the compound of Formula (III) is in a beta form. In some embodiments, the composition includes a combination of an alpha and beta forms of Formula (III). In some embodiments, the compound of Formula (III) is pentagalloyl galactose. In some embodiments, the compound of Formula (III) is pentagalloyl-maltose. In some embodiments, the compound of Formula (III) is pentagalloyl-mannose. In some embodiments, the composition includes a water-soluble solubilizer. In some embodiments, the composition includes a buffer.

Some embodiments relate to a composition including a compound of Formula (IV):

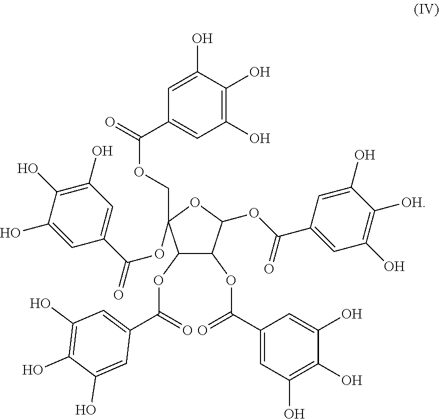 [SMILES: O=C(OCC1(OC(=O)c2cc(O)c(O)c(O)c2)OC(OC(=O)c2cc(O)c(O)c(O)c2)C(OC(=O)c2cc(O)c(O)c(O)c2)C1OC(=O)c1cc(O)c(O)c(O)c1)c1cc(O)c(O)c(O)c1]

In some embodiments, Formula (IV) C1 carbon can be in either alpha, beta, or a mixture of alpha and beta forms. In some embodiments, Formula (IV) is either D- or L-form. In some embodiments, Formula (IV) is an aldopentose. In some embodiments, Formula (IV) is selected from the group consisting of arabinose, lyxose, ribose, or xylose. In some embodiments, the compound of Formula (IV) is in an alpha form. In some embodiments, the compound of Formula (IV) is in a beta form. In some embodiments, the composition includes a combination of an alpha and beta forms of Formula (IV). In some embodiments, the compound of Formula (IV) is pentagalloyl-ribose. In some embodiments, the composition includes a water-soluble solubilizer. In some embodiments, the composition includes a buffer.

Some embodiments relate to a composition including a compound of Formula (V):

wherein, G is galloyl.

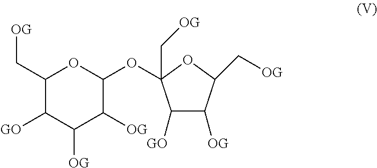

In some embodiments, the compound of Formula (V) is in an alpha form. In some embodiments, the compound of Formula (V) is in a beta form. In some embodiments, the composition includes a combination of an alpha and beta forms of Formula (V). In some embodiments, the composition includes a water-soluble solubilizer. In some embodiments, the composition includes a buffer.

Some embodiments relate to a composition including a compound of Formula (VI):

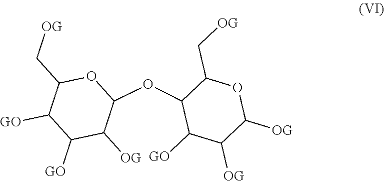

wherein, G is galloyl.

In some embodiments, the compound of Formula (VI) is in an alpha form. In some embodiments, the compound of Formula (VI) is in a beta form. In some embodiments, the composition includes a combination of an alpha and beta forms of Formula (VI). In some embodiments, the composition includes a water-soluble solubilizer. In some embodiments, the composition includes a buffer.

Some embodiments relate to a composition including a compound of Formula (VII):

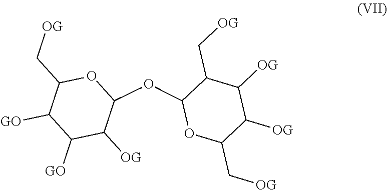

wherein, G is galloyl. In some embodiments, the composition includes a water-soluble solubilizer. In some embodiments, the composition includes a buffer.

In some embodiments, the compound of Formula (VII) is in an alpha form. In some embodiments, the compound of Formula (VII) is in a beta form. In some embodiments, the composition includes a combination of an alpha and beta forms of Formula (VII).

Some embodiments relate to a composition including a compound of Formula (VIII):

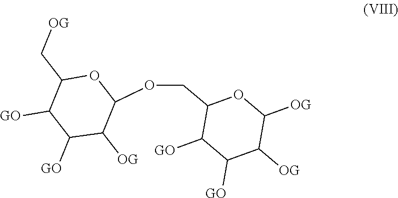

wherein, G is galloyl.

In some embodiments, the compound of Formula (VIII) is in an alpha form. In some embodiments, the compound of Formula (VIII) is in a beta form. In some embodiments, the composition includes a combination of an alpha and beta forms of Formula (VIII). In some embodiments, the composition includes a water-soluble solubilizer. In some embodiments, the composition includes a buffer.

Some embodiments relate to a composition including at least one polygalloyl sugar alcohol. In some embodiments, the at least one polygalloyl sugar alcohol is a sugar alcohol represented by a sugar alcohol represented by the formula (CHOH)nH₂, where n may be 4, 5, or 6 and each hydroxyl group can optionally be substituted with a galloyl group. In some embodiments, each hydroxyl of the at least one polygalloyl sugar alcohol is substituted with a galloyl group. In some embodiments, the at least one polygalloyl sugar alcohol may be selected from, but not limited to, polygalloyl erythritol, polygalloyl threitol, polygalloyl arabitol, polygalloyl xylitol, polygalloylribitol, polygalloyl mannitol, polygalloyl sorbitol, polygalloyl galactitol, polygalloyl fucitol, or polygalloyl iditol, or a combination thereof.

Some embodiments relate to a composition including a compound of Formula (IX):

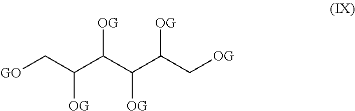

wherein, G is galloyl.

In some embodiments, a compound of Formula (IX) is selected from hexagalloyl sorbitol, hexagalloyl mannitol, hexagalloyl maltitol, hexagalloyl galactitol, hexagalloyl fucitol, or hexagalloyl isomalt. In some embodiments, the composition includes a water-soluble solubilizer. In some embodiments, the composition includes a buffer.

Some embodiments relate to a composition including a compound of Formula (X):

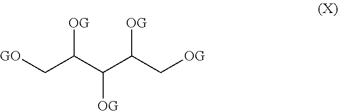

wherein, G is galloyl.

In some embodiments, a compound of Formula (X) is selected from pentagalloyl xylitol, pentagalloyl ribitol, or pentagalloyl arabitol. In some embodiments, the composition includes a water-soluble solubilizer. In some embodiments, the composition includes a buffer.

Some embodiments relate to a composition including a compound of Formula (XI):

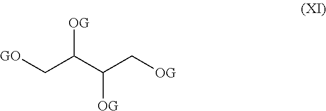

wherein, G is galloyl.

In some embodiments, a compound of Formula (XI) is tetragalloyl erythritol. In some embodiments, the composition includes a water-soluble solubilizer. In some embodiments, the composition includes a buffer.

In some embodiments, the composition includes from about 0.1%, 0.5%, 1%, 2%, 3%, 4%, 5%, (w/v) of a compound of Formula (I), (II), (III), (IV), (V), (VI), (VII), (VIII), (IX), (X), or (XI), or ranges including and/or spanning the aforementioned values. In some embodiments, the formulation includes from about 1.0 mg/ml, 1.3 mg/ml, 1.5 mg/ml, 1.7 mg/ml, 1.9 mg/ml, 2.0 mg/ml, 2.1 mg/ml, 2.3 mg/ml, 2.5 mg/ml, 2.7 mg/ml, 2.9 mg/ml, 3.0 mg/ml, 3.1 mg/ml, 3.3 mg/ml, 3.5 mg/ml, 3.7 mg/ml, 3.9 mg/ml, 4.0 mg/ml Formula (I), (II), (III), (IV), (V), (VI), (VII), (VIII), (IX), (X), or (XI), or ranges including and/or spanning the aforementioned values. In some embodiments, the formulation includes from about 1.5 mg/ml to about 3.5 mg/ml. In some embodiments the formulation includes from about 2.5 mg/ml. In some embodiments, the amount of Formula (I), (II), (III), (IV), (V), (VI), (VII), (VIII), (IX), (X), or (XI), is adjusted for assay, impurities, and loss of drying. In some embodiments, composition does not include pentagalloyl β-glucose. In some embodiments, the compound of Formula (I), (II), (III), (IV), (V), (VI), (VII), or (VIII) is selected from, but not limited to, α-PGG, polygalloyl galactose, polygalloyl maltose, polygalloyl mannose, polygalloyl ribose, polygalloyl sucrose, polygalloyl fructose, polygalloyl inositol, polygalloyl erythritol, or a combination thereof.

In some embodiments, the composition includes from about 0.1%, 0.5%, 1%, 2%, 3%, 4%, 5%, (w/v) of a galloyl ester, or ranges including and/or spanning the aforementioned values. In some embodiments, the galloyl ester may be selected from, but not limited to, α-PGG, polygalloyl galactose, polygalloyl maltose, polygalloyl mannose, polygalloyl ribose, polygalloyl sucrose, polygalloyl-fructose, polygalloyl inositol, polygalloyl erythritol, or a combination thereof. In some embodiments, the galloyl esters may be free of β-PGG.

In some embodiments, the compound of Formula (I), (II), (III), (IV), (V), (VI), (VII), (VIII), (IX), (X), or (XI), is substantially pure. In some embodiments, the compound is at least 95%, 95.5%, 96%, 96.5%, 97%, 97.5%, 98%, 98.5%, 99%, 99.5%, pure, or ranges including and/or spanning the aforementioned values.

In some embodiments, the solubilizer is a water-soluble solubilizer. In some embodiments, the solubilizer may be a contrast agent. In some embodiments, the contrast agent is a radiocontrast material. In some embodiments, the non-ionic solubilizer is a non-ionic iodinated contrast agent. In some embodiments, the non-ionic iodinated contrast agent may act as a radiopaque contrast agent as well as a solubilizer. In some embodiments, the contrast agent is a radiopaque material. In some embodiments, the contrast agent is an ionic contrast agent. In some embodiments, the ionic contrast agent may be selected from, but not limited to, diatrizoate, metrizoate, iothalamate, ioxaglate, or combinations thereof. In some embodiments, the radiocontrast agent is an iodinated contrast agent. In some embodiments, the contrast agent may be a non-ionic iodinated contrast agent. In some embodiments, the non-ionic iodinated contrast agent may be selected from, but not limited to, iopamidol, iohexol, ioxilan, iopromide, iodixanol, iobitridol, ioversol, or combinations thereof. In some embodiments, the composition includes from about 10.0%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35% (w/v) contrast agent, or ranges including and/or spanning the aforementioned values. In some embodiments, the composition includes from about 10.0% to 35% (w/v) contrast agent. In some embodiments, the composition includes from about 22.5% to about 27.5% non-ionic contrast agent. In some embodiments, the composition includes about 24% non-ionic contrast agent. In some embodiments, the composition includes about 24% iopamidol. In some embodiments, the composition includes from about 160 mg/ml, 170 mg/ml, 180 mg/ml, 180 mg/ml, 190 mg/ml, 200 mg/ml, 200 mg/ml, 210 mg/ml, 220 mg/ml, 230 mg/ml, 240 mg/ml, 250 mg/ml, 260 mg/ml, 270 mg/ml, 280 mg/ml, 290 mg/ml, 300 mg/ml, 310 mg/ml, 320 mg/ml, 330 mg/ml, 340 mg/ml, 350 mg/ml, 360 mg/ml preservative, or ranges including and/or spanning the aforementioned values. In some embodiments, the composition includes from about 230 mg/ml to about 290 mg/ml non-ionic contrast agent. In some embodiments, the composition includes about 260 mg/ml non-ionic contrast agent. In some embodiments, the composition includes about 260 mg/ml iopamidol.

In some embodiments, the composition may include an osmolality agent. In some embodiments, the osmolality agent may be selected from, but not limited to sodium chloride, dextrose, mannitol, sucrose, trehalose, propylene glycol, glycerin, or combinations thereof. In some embodiments, the osmolality agent is sodium chloride. In some embodiments, the formulation includes from about 0.2%, 0.25%, 0.3%, 0.35%, 0.4%, 0.45%, 0.5%, 0.55%, 0.6%, 0.65%, 0.7%, 0.75%, 0.8% osmolality agent, or ranges including and/or spanning the aforementioned values. In some embodiments, the composition includes from about 0.45% to about 0.75% osmolality agent. In some embodiments, the composition includes about 0.6% osmolality agent. In some embodiments, the formulation includes about 0.58% sodium chloride. In some embodiments, the composition includes from about 4.0 mg/ml, 4.2 mg/ml, 4.4 mg/ml, 4.6 mg/ml, 4.8 mg/ml, 5.0 mg/ml, 5.2 mg/ml, 5.4 mg/ml, 5.6 mg/ml, 5.8 mg/ml, 6.0 mg/ml, 6.2 mg/ml, 6.4 mg/ml, 6.6 mg/ml, 6.8 mg/ml, 7.0 mg/ml, 7.2 mg/ml, 7.4 mg/ml, 7.6 mg/ml, 7.8 mg/ml, 8.0 mg/ml, osmolality agent, or ranges including and/or spanning the aforementioned values. In some embodiments, the composition includes from about 5.2 mg/ml to about 7.2 mg/ml osmolality agent. In some embodiments, the composition includes about 6.2 mg/ml osmolality agent. In some embodiments, the composition includes about 6.2 mg/ml sodium chloride.

In some embodiments, the composition includes a pH from about 3.0, 3.5, 4.0, 4.5, 5.0, 5.5, 6.0, 6.5, 7.0, 7.5, 8.0, 8.5, 9.0, or ranges including and/or spanning the aforementioned values. In some embodiments, the composition includes a buffer. In some embodiments, the formulation may include one or more buffering agents. In some embodiments, the one or more buffering agents is an acid and its conjugate base. In some embodiments, the one or more buffering agents is a base and its conjugate acid. In some embodiments, the buffer may include, but is not limited to, lactic acid, lactate, citric acid, citrate, carbonic acid, carbonate, phosphate buffer, boronic acid, borate, acetic acid, acetate, or combinations thereof. In some embodiments, the lactic acid is L-lactic acid. In some embodiments, the sodium lactate is L-sodium lactate. In some embodiments, the composition includes a buffer system. In some embodiments, the buffer or buffer system includes, but is not limited to, lactic acid/lactate, citric acid/citrate, carbonic acid/carbonate, phosphate buffer system, boronic acid/borate, acetic acid/acetate, or combinations thereof. In some embodiments, the buffer system is lactic acid/sodium lactate. In some embodiments, the composition includes a buffer or buffer system with a pH from about 3 to about 6. In some embodiments, the composition includes a buffer or buffer system with a pH from about 3 to about 4.5. In some embodiments, the composition includes a buffer or buffer system with a pH from about 3.8.

In some embodiments, the composition may include an excipient. In some embodiments, the excipient is a stabilizer. In some embodiments, the stabilizer is an aminocarboxylate. In some embodiments, the stabilizer may be selected from, but not limited to, N-hydroxyethylaminodiacetic acid, hydroxyethylenediaminetetraacetic acid, nitrilotriacetic acid (NTA), ethylenediaminetetraacetic acid (EDTA), disodium EDTA, N-hydroxyethyl-ethylenediaminetriacetic acid (HEDTA), disodium ethylenediaminetetraacetate (Na₂H₂EDTA), tetrasodium ethylenediaminetetraacetate (Na₄EDTA), and calcium disodium ethylenediaminetetraacetate (CaNa₂EDTA), diethylenetriaminepentaacetic acid (DTPA), and alanine-N,N-diacetic acid, and combinations thereof. In some embodiments, the stabilizer is disodium EDTA. In some embodiments, the composition may include from about 0.4 mg/ml to about 0.6 mg/ml stabilizer. In some embodiments, the composition may include from about 0.4 mg/ml to about 0.6 mg/ml disodium EDTA. In some embodiments, the composition may include from about 0.5 mg/ml disodium EDTA.

In some embodiments, the formulation may include a solvent. In some embodiments, the solvent may include, but is not limited to, water, sterile water for injection, saline water, water and alcohol in a blend, ethanol, dimethylsulfoxide, or combinations thereof. In some embodiments, the solvent is water. In some embodiments, the solvent is sterile water for injection. In some embodiments, the composition may include from about 690 mg/ml to about 890 mg/ml solvent. In some embodiments, the composition may include from about 690 mg/ml to about 890 mg/ml sterile water for injection. In some embodiments, the composition may include from about 790 mg/ml sterile water for injection.

In some embodiments, the composition includes a compound of Formula (I), (II), (III), (IV), (V), (VI), (VII), (VIII), (IX), (X), or (XI), a water-soluble solubilizer, an osmolality agent, a buffer, a stabilizer and a solvent. In some embodiments, the formulation includes an active, a water-soluble solubilizer, an osmolality agent, a buffer system, a stabilizer and a solvent. In some embodiments, the formulation includes from about 0.10% to about 30.0% of a compound of Formula (I), (II), (III), (IV), (V), (VI), (VII), (VIII), (IX), (X), or (XI), about 10% to about 30% non-ionic contrast agent, about 0.5% to about 5.0% osmolality agent, about 0.10% to about 3.0% of an acid, about 0.2% to about 4.0% of a conjugate base of the acid, about 0.04% to about 1.0% stabilizer, and about 50% to about 80% solvent. In some embodiments, the composition includes from about 1.0 mg/ml to about 10.0 mg/ml of a compound of Formula (I), (II), (III), (IV), (V), (VI), (VII), (VIII), (IX), (X), or (XI), about 100 mg/ml to about 400 mg/ml non-ionic contrast agent, about 2 mg/ml to about 12 mg/ml osmolality agent, about 1 mg/ml to about 6 mg/ml of an acid, about 1 mg/ml to about 10 mg/ml of a conjugate base of the acid, about 0.1 mg/ml to about 1.0 mg/ml stabilizer, and about 500 mg/ml to about 1,000 mg/ml solvent. In some embodiments, the formulation includes a compound of Formula (I), (II), (III), (IV), (V), (VI), (VII), (VIII), (IX), (X), or (XI), iopamidol, sodium chloride, lactic acid, sodium lactate, disodium EDTA, and sterile water for injection. In some embodiments, the formulation includes from about 0.10% to about 30.0% of a compound of Formula (I), (II), (III), (IV), (V), (VI), (VII), (VIII), (IX), (X), or (XI), about 22.5% to about 27.5% iopamidol, about 0.45% to about 0.75% sodium chloride, about 0.10% to about 0.30% of lactic acid, about 0.22% to about 0.42% sodium lactate, about 0.04% to about 0.06% disodium EDTA, and about 70% to about 80% sterile water for injection. In some embodiments, composition does not include pentagalloyl β-glucose. In some embodiments, the compound of Formula (I), (II), (III), (IV), (V), (VI), (VII), or (VIII) is selected from, but not limited to, α-PGG, polygalloyl galactose, polygalloyl maltose, polygalloyl mannose, polygalloyl ribose, polygalloyl sucrose, polygalloyl fructose, polygalloyl inositol, polygalloyl erythritol, or a combination thereof.

### Administration and Pharmaceutical Compositions

Administration of any compounds or pharmaceutically active substances disclosed herein or any pharmaceutically acceptable salts thereof or compositions described herein can be administered via any of the accepted modes of administration for agents that serve similar utilities including, but not limited to, orally, subcutaneously, intravenously, intranasally, topically, transdermally, intraperitoneally, intramuscularly, intrapulmonary, vaginally, rectally, or intraocularly.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of a compound described herein (including polymorphs, and solvates thereof), or pharmaceutically acceptable salts thereof; and (b) a pharmaceutically acceptable carrier, diluent, excipient or combination thereof.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, for example, in Gilman et al. (Eds.) (1990); Goodman and Gilman's: The Pharmacological Basis of Therapeutics, 8th Ed., Pergamon Press, which is incorporated herein by reference in its entirety.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (for example, from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for intracerebral, intracranial, intra-arterial, intravenous, intramuscular, or other parental routes of administration. Depending upon the particular route of administration desired, a variety of pharmaceutically acceptable carriers well-known in the art may be used. Pharmaceutically acceptable carriers include, for example, solid or liquid fillers, diluents, hydrotropies, surface-active agents, and encapsulating substances. Optional pharmaceutically active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Compositions described herein may optionally include other drug actives.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

### Methods of Treatment

Aspects of the disclose relate to methods and uses of a compound or composition as described herein to treat elastin-depleted tissues. Some embodiments include methods of treating an aneurysm with and compositions comprising compounds described herein. Some methods include administering a compound, composition, pharmaceutical composition described herein to a subject in need thereof. In some embodiments, a subject can be an animal, for example, a mammal, a human. In some embodiments, the subject is a human.

Further embodiments include administering a combination of compounds as described herein to a subject in need thereof. In some embodiments, a combination can include a compound, composition, pharmaceutical composition described herein with an additional medicament.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially.

In some embodiments, a composition as described herein may be delivered to an elastin-depleted tissue. In some embodiments, the compound or composition as described herein may be provided to treat a disease or condition associated with an elastin-depleted tissue. In some embodiments, the compound or composition as described herein may be provided with a device as described herein to the elastin-depleted tissue thereby treating the elastin-depleted tissue. In some embodiments, a method for treating, preventing, and/or slowing the growth of elastin-depleted tissue includes administering a therapeutically effective amount of a compound or composition as described herein to a subject in need, thereby treating, preventing, and/or slowing the growth of elastin-depleted tissue.

In some embodiments, a composition as described herein may be delivered to the blood vessel wall for treatment of an aneurysm, such as an abdominal aortic aneurysm. Without being limited by theory, the delivery of a compound of Formula (I), (II), (III), (IV), (V), (VI), (VII), (VIII), (IX), (X), (XI), or a pharmaceutically acceptable salt thereof, to the blood vessel wall where an aneurysm has formed may stabilize the aneurysm by cross-linking, at least transiently, the elastin proteins within the extracellular matrix of the connective tissue of the blood vessel wall. In some embodiments, treatment of the blood vessel with an elastin-stabilizing compound, such as those described herein, may increase the mechanical integrity of the blood vessel where the aneurysm is present. In some embodiments, treatment with a compound as described herein may prevent, inhibit, and/or slow the growth of an aneurysm and further thinning of the blood vessel wall and may prevent, inhibit, reduce the likelihood of, and/or delay the risk of rupture of the aneurysm. In some instances, treatment with a compound as described herein may facilitate natural healing of the aneurysm by mechanically stabilizing the aneurysm. In some embodiments, treatment with a compound as described herein may be used prior to, after, and/or concurrently with other interventional treatment of an aneurysm, such as surgical intervention. In some embodiments, treatment with a compound of a compound described herein may be particularly suitable for treating abdominal aortic aneurysms between approximately 4-5 cm in diameter.

In some embodiments, a method of treating an aneurysm in a subject in need thereof includes administering a compound or composition as described herein. A subject in need of receiving a compound or composition as described herein may not always be identified prior to receiving a first treatment with the formulation. For example, a subject may be predetermined that they will develop an aneurysm prior to showing signs of an aneurysm. Alternatively, the subject may receive treatment prophylactically if he or she is at risk or not at risk of an aneurysm. Accordingly, in some embodiments, the compound or composition is administered to the subject after the subject receives an early-stage diagnosis. In some embodiments, not every subject is a candidate for such administration and identification of treatment subjects may be desirable. It is understood that patient selection depends upon a number of factors within the skill of the ordinarily skilled physician. Thus, some embodiments disclosed herein further comprise identifying a subject as one that will benefit from administering an effective amount of a compound or composition described herein to treat an aneurysm. Subjects may be identified on the basis of physiological factors specific to the subject according to the subject's age, present medical condition, present medical treatment, prescribed medical treatment, or in some embodiments, the subject being diagnosed with comorbidities associated with an aneurysm. In some embodiments, treatment of an aneurysm includes preventing, reducing, and/or slowing a bulge or weakened area in the wall of a blood vessel.

Some embodiments include methods of treating, preventing, reducing, or ameliorating an abdominal aortic aneurysm (AAA) by administering a therapeutically effective amount of a compound or composition as described herein to a subject in need thereof. In some embodiments, administration of the compound or composition as described herein prevents the rupture of the AAA. In some embodiments, administration of the compound or composition as described herein reduces the thinning of the wall of the blood vessel. In some embodiments, administration of the formulation prevents the AAA from leaking. In some embodiments, administration of the compound or composition as described herein prevents life-threatening bleeding from the AAA.

Some embodiments include methods of treating, preventing, reducing, or ameliorating a thoracic aortic aneurysm (TAA) by administering a therapeutically effective amount of a compound or composition as described herein as described herein to a subject in need thereof. In some embodiments, administration of the compound or composition as described herein prevents the rupture of the TAA. In some embodiments, administration of the compound or composition as described herein reduces the thinning of the wall of the blood vessel. In some embodiments, administration of the compound or composition as described herein prevents the TAA from leaking. In some embodiments, administration of the compound or composition as described herein prevents life-threatening bleeding from the TAA.

Some embodiments include methods of treating, preventing, reducing, or ameliorating a peripheral aneurysm by administering a therapeutically effective amount of a compound or composition as described herein as described herein to a subject in need thereof. In some embodiments, administration of the compound or composition as described herein prevents the rupture of the peripheral aneurysm. In some embodiments, administration of the compound or composition as described herein reduces the thinning of the wall of the blood vessel. In some embodiments, administration of the compound or composition as described herein prevents the peripheral aneurysm from leaking. In some embodiments, administration of the compound or composition as described herein prevents life-threatening bleeding from the peripheral aneurysm.

In some embodiments, a composition as described herein may be used to treat aneurysms besides abdominal aortic aneurysms, including peripheral and neural aneurysms. In some embodiments, the compound or composition as described herein may be delivered to these aneurysms by the same device as described herein with respect to abdominal aortic aneurysms or one similar thereto or may be delivered using another device or route of administration. For instance, in some embodiments, a compound or composition as described herein, particularly a high purity a compound described herein, may be suitable for direct injection into the bloodstream or into another tissue for treatment of other indications. In some embodiments, the compounds or composition as described herein may be used to coat vascular stents and/or grafts.

Some embodiments relate to a method for treating, preventing, ameliorating, or reducing an aortopathic condition or disease in a subject in need thereof. In some embodiments, the method includes administering a composition comprising one or more polygalloyl compound, a water-soluble solubilizer, and a buffer, wherein the one or more polygalloyl compound is not pentagalloyl β-glucose (β-PGG). In some embodiments, the one or more polygalloyl compound is selected from the group consisting of α-pentagalloyl glucose, pentagalloyl galactose, pentagalloyl maltose, pentagalloyl mannose, pentagalloyl ribose, pentagalloyl sucrose, pentagalloyl-fructose, pentagalloyl-inositol, polygalloyl-sucrose, hexagalloyl sorbitol, hexagalloyl mannitol, nonagalloyl maltitol, nonagalloyl isomalt, pentagalloyl xylitol, tetragalloyl erythritol, or combinations thereof. In some embodiments, the aortopathic condition or disease is an aneurysm. In some embodiments, the aneurysm is selected from an aortic aneurysm, a peripheral aneurysm, abdominal aortic aneurysm, or a neuro aneurysm. In some embodiments, the aortic aneurysm is greater than 3 cm in diameter. In some embodiments, the composition is administered by local delivery. In some embodiments, the local delivery is intraluminal delivery. In some embodiments, a site of the administration is accessed via a femoral artery of the subject. In some embodiments, the method further comprises administering a contrast agent prior to administering the composition to the subject. In some embodiments, a site of the administration is accessed via an aortic artery. In some embodiments, the aneurysm is an abdominal aortic aneurysm. In some embodiments, the composition is administered by a catheter device. In some embodiments, the catheter device is about 20 cm to about 50 cm in length. In some embodiments, the catheter device is introduced through an access sheath. In some embodiments, the catheter device further includes at least one guidewire. In some embodiments, the at least one guidewire is received by a first central lumen.

Some embodiments relate to a method for blood vessel elastin stabilization in a subject in need thereof. In some embodiments, the method includes administering a composition comprising one or more polygalloyl compound, or a salt thereof, a water-soluble solubilizer, and a buffer, wherein the one or more polygalloyl compound is not pentagalloyl β-glucose. In some embodiments, the one or more polygalloyl compound is selected from the group consisting of α-pentagalloyl glucose, pentagalloyl galactose, pentagalloyl maltose, pentagalloyl mannose, pentagalloyl ribose, pentagalloyl sucrose, pentagalloyl-fructose, pentagalloyl-inositol, polygalloyl-sucrose, hexagalloyl sorbitol, hexagalloyl mannitol, hexagalloyl maltitol, hexagalloyl isomalt pentagalloyl xylitol, butagalloyl erythritol, or combinations thereof. In some embodiments, the blood vessel elastin stabilization improves an aneurysm in the subject. In some embodiments, the aneurysm is selected from an aortic aneurysm, a peripheral aneurysm, abdominal aortic aneurysm, or a neuro aneurysm. In some embodiments, the aortic aneurysm is greater than 3 cm in diameter. In some embodiments, the composition is administered by local delivery. In some embodiments, the local delivery is intraluminal delivery. In some embodiments, a site of the administration is accessed via a femoral artery of the subject. In some embodiments, the method further comprises administering a contrast agent prior to administering the composition to the subject. In some embodiments, a site of the administration is accessed via an aortic artery. In some embodiments, the aneurysm is an abdominal aortic aneurysm. In some embodiments, the composition is administered by a catheter device. In some embodiments, the catheter device is about 20 cm to about 50 cm in length. In some embodiments, the catheter device is introduced through an access sheath. In some embodiments, the catheter device further includes at least one guidewire. In some embodiments, the at least one guidewire is received by a first central lumen.

### Kits for Delivery

Aspects of the disclosure provide for a compound or composition as described herein may be prepared in a solution for delivery as a therapeutic agent to a patient. In some embodiments, the compound or composition as described may be dissolved in a solubilizer for subsequent delivery to a patient. In some embodiments, the compound or composition as described herein may ultimately be dissolved into a non-toxic aqueous solution suitable for delivery to a patient. The aqueous solution may be a saline solution, as is known in the art, or another aqueous solution comprising salts configured to maintain physiological equilibrium with the intravascular environment. In some embodiments, the compound or composition as described herein may be dissolved into other aqueous solutions. In some embodiments, the solubilized solution including the compound or composition may be warmed (e.g., to above room temperature or above physiological temperature) to dissolve or help dissolve the compound as described herein (or other therapeutic agents). For instance, the saline may be warmed to at least about 25° C., 30° C., 35° C., 40° C., 45° C., 50° C., 55° C., or 60° C. prior to dissolving the compound as described herein. In some implementations, the therapeutic solution may be raised to and/or maintained at an elevated temperature (e.g., physiological temperature) during delivery.

In some embodiments, the compound or composition as described herein (for example, a purified compound as described herein) for a therapeutic treatment, including but not limited to those described elsewhere herein, may be provided in a kit comprising the components necessary to prepare the compound or composition as described herein for delivery in a therapeutic solution. In some embodiments, the kit may comprise the compound as described herein in a solid (dry) form, the solubilizer, and/or the saline solution. The kit may be configured to optimize the storage conditions of the compound as described herein, for short or long-term storage. In some embodiments, the kit may be configured to store the compound as described herein for up to at least 1 week, 2 weeks, 3 weeks, 4 weeks, 1 month, 2 months, 3 months, 4 months, 5 months, 6 months, 1 year, 2 years, or 3 years. The kit may comprise one or more aliquots of each component in pre-measured amounts or volumes. Each component may be provided in a sealed vial, tube, or other container as is known in the art. The containers may each comprise plastic and/or glass. The containers may be configured (for example, tinted or covered) to protect the components from light and/or other radiation. In some embodiments, the kit may be configured for shipping. For example, the components may be contained in a box or other container including desiccants and/or may be configured for temperature control. In some embodiments, the compound or composition as described herein and/or other components may be supplied in a container that has been purged of air (particularly, oxygen). The component may be stored under vacuum or may be purged with an inert gas, such as nitrogen or argon. In some embodiments, the compound as described herein may be mixed with an antioxidant or other stabilizer, in addition to or alternatively to purging the air. In some embodiments, the volume of saline provided may be configured to prepare the compound or composition as described herein at a desired therapeutic concentration. In some embodiments, the volume of saline may be configured to prepare the compound as described herein at a maximal therapeutic concentration, such that a user may dilute the compound or composition as described herein with additional solvent to the desired therapeutic concentration. In some embodiments, the total volume of saline may be configured to prepare the compound as described herein at a concentration below the desired concentration and the user may use only a portion of the volume of the saline to prepare the compound as described herein to the desired concentration. The container of saline may have volume indicators for facilitating measurement of the saline. In some embodiments, the saline may be provided in a plurality of aliquots having the same and/or different volumes, which may allow the user to select an aliquot of a desired volume to prepare the compound as described herein at a desired concentration and/or combine various volumes to prepare the compound as described herein at a desired concentration. In some embodiments, the kit may comprise one or more additional components. For example, the kit may comprise a contrast agent for mixing with the therapeutic the compound as described herein in a solution for allowing indirect visualization of the therapeutic solution, as described elsewhere herein.

Accordingly, some aspects described herein relate to the following numbered alternatives:
1. A method of reducing or inhibiting growth of an aneurysm in a subject having an aneurysm, the method comprising: administering a composition comprising at least one polygalloyl compound, or a salt thereof, a water-soluble solubilizer, and a buffer, wherein the polygalloyl compound is not pentagalloyl β-glucose (β-PGG).
2. The method of Alternative 1, wherein the aneurysm is selected from an aortic aneurysm, a peripheral aneurysm, abdominal aortic aneurysm, or a neuro aneurysm.
3. The method of Alternative 2, wherein the aortic aneurysm is greater than 3 cm in diameter.
4. The method of any one of Alternatives 1 to 3, wherein the composition is administered by local delivery.
5. The method of Alternative 4, wherein the local delivery is intraluminal delivery.
6. The method of Alternative 2, wherein the aneurysm is an abdominal aortic aneurysm.
7. The method of any one of Alternatives 1 to 6, wherein the composition is administered by a catheter device.
8. The method of any one of Alternatives 1 to 7, wherein the at least one polygalloyl compound is fully gallic acid substituted monosaccharides or disaccharides with the proviso that the one or more polygalloyl compound is not β-PGG.
9. The method of any one of Alternatives 1 to 7, wherein the at least one polygalloyl compound is a fully gallic acid substituted disaccharides.
10. The method of any one of Alternatives 1 to 7, wherein the at least one polygalloyl compound is a fully gallic acid substituted sugar alcohol.
11. The method of any one of Alternatives 1 to 10, wherein the polygalloyl compound is selected from the group consisting of pentagalloyl α-glucose, pentagalloyl galactose, pentagalloyl maltose, pentagalloyl mannose, pentagalloyl ribose, pentagalloyl sucrose, pentagalloyl-fructose, pentagalloyl-inositol, polygalloyl-sucrose, hexagalloyl sorbitol, hexagalloyl mannitol, hexagalloyl maltitol, hexagalloyl isomalt pentagalloyl xylitol, tetragalloyl erythritol, or combinations thereof.
12. The method of any one of Alternatives 1 to 7, wherein the at least one polygalloyl compound is a compound of Formula (III):

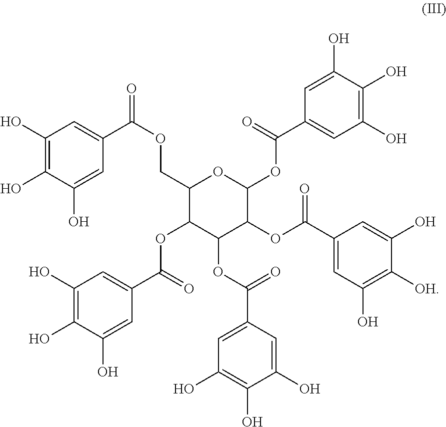 [SMILES: O=C(OCC1OC(OC(=O)c2cc(O)c(O)c(O)c2)C(OC(=O)c2cc(O)c(O)c(O)c2)C(OC(=O)c2cc(O)c(O)c(O)c2)C1OC(=O)c1cc(O)c(O)c(O)c1)c1cc(O)c(O)c(O)c1]

13. The method of Alternative 12, wherein Formula (III) C1 carbon can be in either alpha, beta, or a mixture of the two forms and is not β-PGG.
14. The method of Alternative 12 or 13, wherein Formula (III) is either D- or L-form.
15. The method of any one of Alternatives 12 to 14, wherein Formula (III) is selected from the group consisting of allose, altrose, mannose, gulose, idose, galactose, glucose, or talose.
16. The method of any one of Alternatives 12 to 15, wherein the compound of is selected from the group consisting of pentagallyol α-glucose, pentagalloyl galactose, or pentagalloyl-mannose.
17. The method of any one of Alternatives 12 to 14, wherein the at least one polygalloyl compound is a compound of Formula (IV):

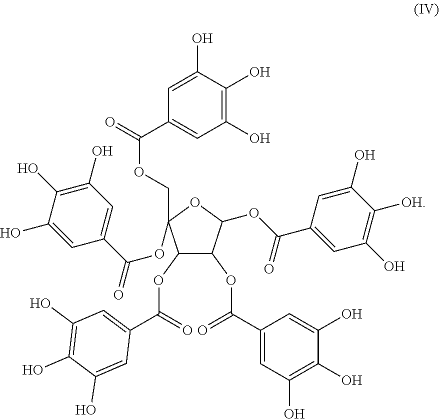 [SMILES: O=C(OCC1(OC(=O)c2cc(O)c(O)c(O)c2)OC(OC(=O)c2cc(O)c(O)c(O)c2)C(OC(=O)c2cc(O)c(O)c(O)c2)C1OC(=O)c1cc(O)c(O)c(O)c1)c1cc(O)c(O)c(O)c1]

18. The method of Alternative 17, wherein Formula (IV) C1 carbon can be in either alpha, beta, or a mixture of alpha and beta forms.
19. The method of Alternative 17 or 18, wherein Formula (IV) is either D- or L-form.
20. The method of any one of Alternatives 17 to 19, wherein Formula (IV) is an aldopentose.
21. The method of Alternative 17 to 20, wherein Formula (IV) is selected from the group consisting of arabinose, lyxose, ribose, or xylose.
22. The method of any one of Alternatives 17 to 21, wherein the compound is pentagalloyl-ribose.
23. The method of any one of Alternatives 1 to 7, wherein the at least one polygalloyl compound is a compound of Formula (II):

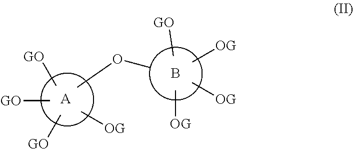

wherein, Ring A and B are each independently selected from a 5-membered ring carbohydrate or a 6-carbon ring carbohydrate; G is galloyl group, wherein, each —OH of the carbohydrate are substituted with OG.
24. The method of Alternative 23, wherein Ring A is an aldohexose, Ring B is an aldohexose, or Ring A and Ring B are each an aldohexose.
25. The method of Alternative 24, wherein the aldohexose is D-form.
26. The method of Alternative 24, wherein the aldohexose is L-form.
27. The method of Alternative 24, wherein the aldohexose is a combination of D- and L-form.
28. The method of any one of Alternatives 24 to 27, wherein the aldohexose is selected from the group consisting of allose, altrose, mannose, gulose, idose, galactose, glucose, or talose.
29. The method of Alternative 23, wherein Ring A is a ketohexose, Ring B is a ketohexose, or Ring A and Ring B are each a ketohexose.
30. The method of Alternative 29, wherein the ketohexose is D-form.
31. The method of Alternative 29, wherein the ketohexose is L-form.
32. The method of Alternative 29, wherein the ketohexose is a combination of D- and L-form.
33. The method of any one of Alternatives 29 to 32, wherein the ketohexose is selected from the group consisting of psicose, fructose, sorbose, or tagatose.
34. The method of Alternative 23, wherein Ring A is an aldopentose, Ring B is an aldopentose, or Ring A and Ring B are each aldopentose.
35. The method of Alternative 34, wherein the aldopentose is D-form.
36. The method of Alternative 34, wherein the aldopentose is L-form.
37. The method of Alternative 34, wherein the aldopentose is a combination of D- and L-form.
38. The method of any one of Alternatives 33 to 37, wherein the aldopentose is selected from the group consisting of arabinose, lyxose, ribose, or xylose.
39. The method of any one of Alternatives 23 to 28, wherein the compound is polygalloyl-sucrose.
40. The composition of Alternative 23, wherein the 5-membered ring carbohydrate or a 6-carbon ring carbohydrate of Formula (II) are each independently selected from the group consisting of ribose, arabinose, xylose, lyxose, allose, altrose, mannose, gulose, idose, galactose, glucose, talose, psicose, fructose, sorbose, and tagatose.
41. The method of Alternative 23 to 40, wherein the 5-membered ring carbohydrate or 6-carbon ring carbohydrate of Formula (II) are each independently in alpha form.
42. The method of Alternative 23 to 40, wherein the 5-membered ring carbohydrate or 6-carbon ring carbohydrate of Formula (II) are each independently in beta form.
43. The method of Alternative 23 to 40, wherein the 5-membered ring carbohydrate or 6-carbon ring carbohydrate of Formula (II) are each independently selected as alpha form, beta form, or a combination thereof.
44. The method of any one of Alternatives 23 to 43, represented by Formula (V):

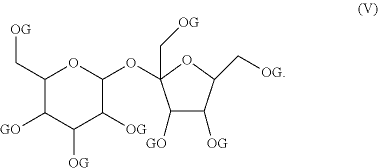

45. he method of any one of Alternatives 23 to 43, represented by Formula (VI):

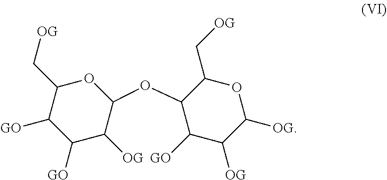

46. The method of any one of Alternatives 23 to 43, represented by Formula (VII):

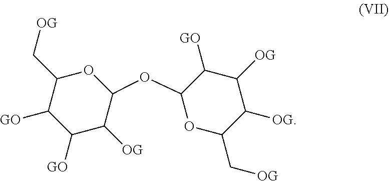

47. The method of any one of Alternatives 23 to 43, represented by Formula (VIII):

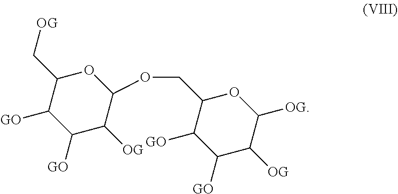

48. The method of any one of Alternatives 1 to 7, wherein the polygalloyl compound is a polygalloyl sugar alcohol.
49. The method of Alternative 48, wherein the polygalloyl sugar alcohol is a sugar alcohol represented by the formula (CHOH)nH₂, where n is 4, 5, or 6, wherein each hydroxyl group is optionally substituted with a galloyl group.
50. The method of Alternative 49, wherein each hydroxyl of the polygalloyl sugar is substituted with a galloyl group.
51. The method of any one of Alternatives 48 to 50, wherein the polygalloyl sugar alcohol is selected from the group selected from consisting of polygalloyl erythritol, polygalloyl threitol, polygalloyl arabitol, polygalloyl xylitol, polygalloylribitol, polygalloyl mannitol, polygalloyl sorbitol, polygalloyl galactitol, polygalloyl fucitol, or polygalloyl iditol, or a combination thereof.
52. The method of any one of Alternatives 1 to 7, wherein the polygalloyl compound is a compound of Formula (IX):

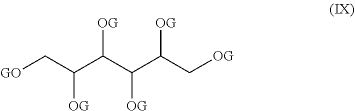

wherein, G is galloyl.
53. The method of any one of Alternatives 1 to 7, wherein the polygalloyl compound is a compound of Formula (X):

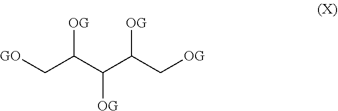

wherein, G is galloyl.
54. The method of any one of Alternatives 1 to 7, wherein the polygalloyl compound is a compound of Formula (XI):

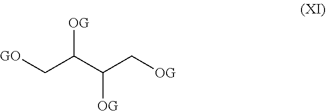

wherein, G is galloyl.
55. The method of any one of Alternatives 1 to 54, further comprising one or more galloyl esters.
56. The method of any one of Alternatives 1 to 54, wherein the composition is substantially free of gallic acid.
57. The method of any one of Alternatives 1 to 56, wherein substantially free of methyl gallate.
58. The method of any one of Alternatives 1 to 54, wherein the water-soluble solubilizer is a contrast agent.
59. The method of Alternative 58, wherein the contrast agent is a radiocontrast material.
60. The method of Alternative 58, wherein the contrast agent is a radiocontrast material is a non-ionic radio-opaque contrast agent.
61. The method of Alternative 60, wherein the non-ionic radio-opaque contrast agent is selected from the group consisting of iopamidol, iohexol, iodixanol, or a combination thereof.
62. The method of any one of Alternatives 1 to 59, wherein the water-soluble solubilizer is from about 10 to about 30% (w/v) of the composition.
63. The method of any one of Alternatives 1 to 62, wherein the buffer is selected from the group consisting of lactic acid/lactate, citric acid/citrate, carbonic acid/carbonate, phosphate buffer system, boronic acid/borate, acetic acid/acetate, or combinations thereof.
64. The method of any one of Alternatives 1 to 62, wherein the composition is at a pH between 2 to 6.
65. The method of Alternative 64, wherein the composition is at a pH between 3 to 5.
66. The method of any one of Alternatives 1 to 65, further comprising an osmolality agent.
67. The method of Alternative 66, wherein the osmolality agent is selected from potassium chloride, calcium chloride, sodium lactate, sodium chloride, dextrose, mannitol, sucrose, trehalose, propylene glycol, glycerin, or combinations thereof.
68. The method of any one of Alternatives 1 to 67, further comprising N-hydroxyethylaminodiacetic acid, hydroxyethylenediaminetetraacetic acid, nitrilotriacetic acid (NTA), ethylenediaminetetraacetic acid (EDTA), disodium EDTA, N-hydroxyethyl-ethylenediaminetriacetic acid (HEDTA), disodium ethylenediaminetetraacetate (Na₂H₂EDTA), tetrasodium ethylenediaminetetraacetate (Na₄EDTA), and calcium disodium ethylenediaminetetraacetate (CaNa₂EDTA), diethylenetriaminepentaacetic acid (DTPA), and alanine-N,N-diacetic acid, and combinations thereof.
69. The method of Alternative 4 or 5, wherein the local delivery comprises a stent coated by the composition according to any one of Alternatives 12 to 68.
70. The method of Alternative 4 or 5, wherein the local delivery comprises a balloon coated by the composition according to any one of Alternatives 12 to 68.
71. he method of Alternative 4 or 5, wherein the local delivery comprises a weeping balloon by the composition according to any one of Alternatives 12 to 68.
72. The method of Alternative 4 or 5, wherein the local delivery comprises a catheter device by the composition according to any one of Alternatives 12 to 68.
73. A method for treating, preventing, ameliorating, or reducing an aortopathic condition or disease in a subject in need thereof, the method comprising administering a composition comprising a polygalloyl compound, or a salt thereof, a water-soluble solubilizer, and a buffer, wherein the polygalloyl compound is not pentagalloyl β-glucose (β-PGG).
74. The method of Alternative 73, wherein the polygalloyl compound is selected from the group consisting of α-pentagalloyl glucose, pentagalloyl galactose, pentagalloyl maltose, pentagalloyl mannose, pentagalloyl ribose, pentagalloyl sucrose, pentagalloyl-fructose, pentagalloyl-inositol, polygalloyl-sucrose, hexagalloyl sorbitol, hexagalloyl mannitol, hexagalloyl maltitol, hexagalloyl isomalt pentagalloyl xylitol, tetragalloyl erythritol, or combinations thereof.
75. The method of Alternative 73 or 74, wherein the aortopathic condition or disease is an aneurysm.
76. The method of Alternative 75, wherein the aneurysm is selected from an aortic aneurysm, a peripheral aneurysm, abdominal aortic aneurysm, or a neuro aneurysm.
77. The method of Alternative 76, wherein the aortic aneurysm is greater than 3 cm in diameter.
78. The method of any one of Alternatives 73 to 77, wherein the composition is administered by local delivery.
79. The method of Alternative 78, wherein the local delivery is intraluminal delivery.
80. The method of any one of Alternatives 73 to 79, wherein a site of the administration is accessed via a femoral artery of the subject.
81. The method of any one of Alternatives 73 to 80, wherein the method further comprises administering a contrast agent prior to administering the composition to the subject.
82. The method of any one of Alternatives 73 to 81, wherein a site of the administration is accessed via an aortic artery.
83. The method of Alternative 75, wherein the aneurysm is an abdominal aortic aneurysm.
84. The method of any one of Alternatives 73 to 83, wherein the composition is administered by a catheter device.
85. The method of Alternative 84, wherein the catheter device is about 20 cm to about 50 cm in length.
86. The method of Alternative 84 to 85, wherein the catheter device is introduced through an access sheath.
87. The method of any one of Alternatives 83 to 85, wherein the catheter device further includes at least one guidewire.
88. The method of Alternative 86, wherein the at least one guidewire is received by a first central lumen.
89. A method for blood vessel elastin stabilization in a subject in need thereof, the method comprising, administering a composition comprising a polygalloyl compound, or a salt thereof, a water-soluble solubilizer, and a buffer, wherein the polygalloyl compound is not pentagalloyl β-glucose (β-PGG).
90. The method of Alternative 89, wherein the polygalloyl compound is selected from the group consisting of α-pentagalloyl glucose, pentagalloyl galactose, pentagalloyl maltose, pentagalloyl mannose, pentagalloyl ribose, pentagalloyl sucrose, pentagalloyl-fructose, pentagalloyl-inositol, polygalloyl-sucrose, hexagalloyl sorbitol, hexagalloyl mannitol, hexagalloyl maltitol, hexagalloyl isomalt pentagalloyl xylitol, butagalloyl erythritol, or combinations thereof.
91. The method of Alternative 89 or 90, wherein the blood vessel elastin stabilization improves an aneurysm in the subject.
92. The method of Alternative 91Error! Reference source not found., wherein the aneurysm is selected from an aortic aneurysm, a peripheral aneurysm, abdominal aortic aneurysm, or a neuro aneurysm.
93. The method of Alternative 91, wherein the aortic aneurysm is greater than 3 cm in diameter.
94. The method of any one of Alternatives 89 to 93, wherein the composition is administered by local delivery.
95. The method of Alternative 94, wherein the local delivery is intraluminal delivery.
96. The method of any one of Alternatives 89 to 95, wherein a site of the administration is accessed via a femoral artery of the subject.
97. The method of any one of Alternatives 89 to 96, wherein the method further comprises administering a contrast agent prior to administering the composition to the subject.
98. The method of any one of Alternatives 89 to 97, wherein a site of the administration is accessed via an aortic artery.
99. The method of Alternative 98, wherein the aneurysm is an abdominal aortic aneurysm.
100. The method of any one of Alternatives 89 to 99, wherein the composition is administered by a catheter device.
101. The method of Alternative 100, wherein the catheter device is about 20 cm to about 50 cm in length.
102. The method of Alternative 100 or 101, wherein the catheter device is introduced through an access sheath.
103. The method of any one of Alternatives 89 to 102, wherein the catheter device further includes at least one guidewire.
104. The method of Alternative 103, wherein the at least one guidewire is received by a first central lumen.

### EXAMPLES

To further illustrate inventive aspects of the present disclosure, the following examples are included. The examples should not, of course, be construed as specifically limiting the disclosure. Variations of these examples within the scope of the claims are within the purview of one skilled in the art and are considered to fall within the scope of the invention as described and claimed herein. The reader will recognize that the skilled artisan, armed with the present disclosure, and skill in the art is able to prepare and use the inventive concepts without exhaustive examples.

### General Procedures

Additional embodiments are disclosed in further detail in the following examples, which are not in any way intended to limit the scope of the claims. Materials used in preparing compounds of Formula (I), (la), (lb), or (Ic), described herein may be made by known methods or are commercially available. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March's Advanced Organic Chemistry (Wiley), Carey and Sundberg, Advanced Organic Chemistry (incorporated herein by reference in their entirety) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application. Trademarks used herein are examples only and reflect illustrative materials used at the time of the filing of the present application. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments.

The following example schemes are provided for the guidance of the reader, and collectively represent an example method for making the compounds provided herein. Furthermore, other methods for preparing compounds described herein will be readily apparent to the person of ordinary skill in the art in light of the following reaction schemes and examples. Unless otherwise indicated, all variables are as defined above.

### Example 1—General Synthesis A

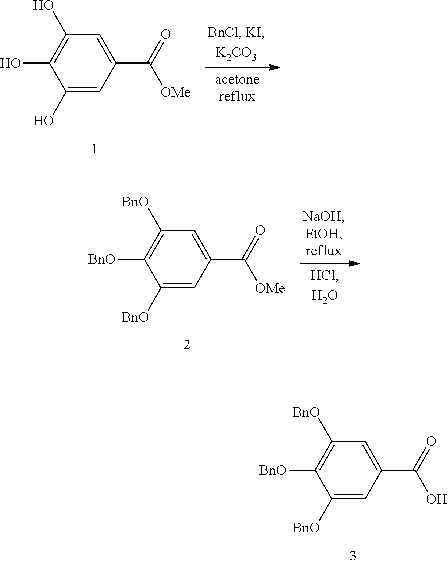 [SMILES: COC(=O)c1cc(O)c(O)c(O)c1 ; COC(=O)c1cc(OCc2ccccc2)c(OCc2ccccc2)c(OCc2ccccc2)c1 ; O=C(O)c1cc(OCc2ccccc2)c(OCc2ccccc2)c(OCc2ccccc2)c1]

Compound 2. Five hundred grams of gallic acid methyl ester (Compound 1) was charged by adding K₂CO₃ (6.0 eq) and KI (0.5 eq) to Compound 1 and mixed in acetone (15 V) at 20° C. The mixture was stirred for 30 minutes at 20° C. BnCl (3.2 eq) was drop wised added at 20° C. within 30 minutes. The mixture was stirred for 18 hours at 60° C. The reaction was monitored by LCMS, P: 80.7% SM: 0%. The reaction was cooled to 20° C. The reaction was filtrated and the cake was washed with DCM (2 L×3). The reaction concentration was set to 1 V remaining, charged MTBE (5 L). The reaction was stirred for 2 hours at 20° C. The reaction was filtrated, and the cake was washed with MTBE (500 ml×2). The cake was dried and resulted in 810 g of Compound 2.

Compound 3. Eight hundred ten grams of Compound 2 was charged by adding ethanol to Compound 2 and mixing at 10V at 20° C. The suspension mixture was stirred for 10 minutes at 20° C. Sodium hydroxide (1.5 eq was dissolved in 2 V H₂O) was added to the mixture. The mixture was stirred for 2 hours at 80° C. The reaction was monitored by LCMS, P: 99.6%. The reaction was then cooled to 20° C. The mixture was poured into 0.6 M HCl (1.1 L). The mixture was stirred for 30 minutes at 20° C. The mixture was filtrated and the cake was washed with EtOH:H₂O (1:1, 5 L×2). The solid was washed with MeOH (1 L×1) and MTBE (5 L×2). The cake was dried and produced 702 g of Compound 3.

### Example 2—General Synthesis B

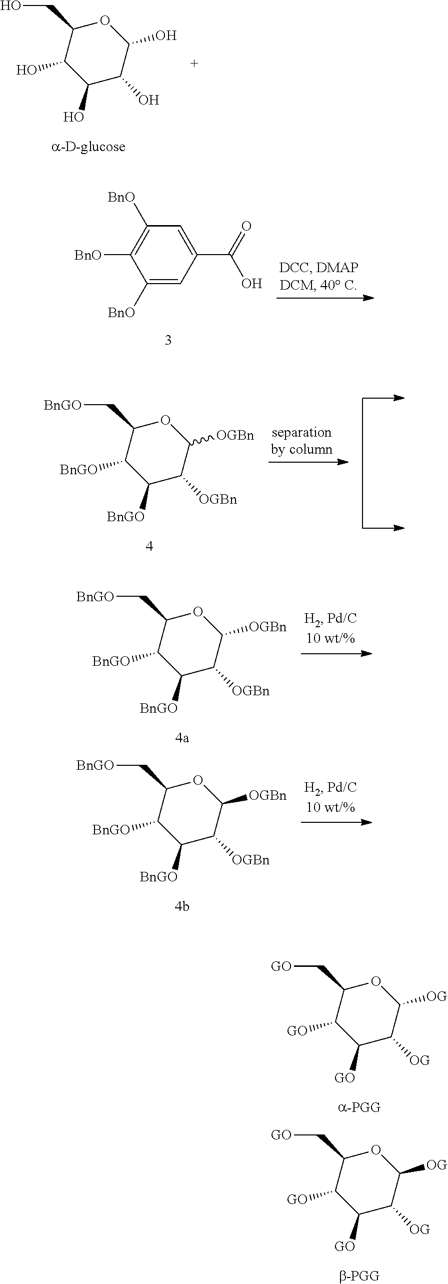

Compound 4. α-D-glucose (1.0 eq), DCM, and Compound 3 (7.3 eq) was charged at 30 V at 20° C. DMAP (8.0 eq) and DCC (9.0 eq) was charged and added to the mixture and stirred for 10 minutes at 20° C. The mixture was stirred for 16 hours at 40° C. The reaction was monitored by LCMS. The reaction was cooled to 20° C. The reaction was filtrated and the cake was washed with DCM (10 V). The filtrate was concentrated, and purified by silica gel column by EA:PE (1:100). The reactant was concentrated and the crude was used to the next step directly.

α-PGG. Compound 4a was stirred for 16 hours at 60° C. under H₂ atmosphere (10 atm). The reaction was filtrated and the cake was washed with MeOH (10 V). The reactant was concentrated and purified by Prep-HPLC and identified as α-PGG. The purity was 97.89%, and 78.78% QNMR. [R]24D+167.06° (c 0.17, CH₃COCH₃); IR (KBr) 3390, 1699, 1612, 1535 cm⁻¹; HRMS (ESI) (C41H32O26), m/z calculated M-H+ 939.1104, measured M-H+ 939.1107 (0.3 ppm); ¹H NMR (CD3COCD3) δ 4.38 (1H, dd, J) 4.0, 13.0), 4.5 (1H, dd, J) 1.5, 11.0), 4.66 (1H, d, J) 9.5), 5.45 (1H, dd, J) 3.5, 9.0), 5.76 (1H, t, J) 10.5),6.16 (1H, t, J) 10.5), 6.72 (1H, d, J) 3.5), 6.90 (2H, s), 6.91 (2H, s), 7.02 (2H, s), 7.18 (2H, s), 7.22 (2H, s).

β-PGG. Compound 4b was stirred for 16 hours at 60° C. under H₂ atmosphere (10 atm). The reaction was filtrated and the cake was washed with MeOH (10 V). The reactant was concentrated and purified by Prep-HPLC and identified as β-PGG. The purity was 99.01%. [R]24D+17.14° (c 0.14, CH₃COCH₃); IR (KBr) 3392, 1708, 1616, 1535 cm⁻¹; HRMS (ESI) (C41H32O26), m/z calculated M-H+ 939.1104, measured M-H+ 939.1075 (3.1 ppm); ¹HNMR (CD₃COCD₃) δ 4.36 (1H, dd, J 5.0, 13.0), 4.52 (1H, m), 4.54 (1H, m), 5.60 (1H, t, J 9.5), 5.64 (1H, t, J 10.0), 5.99 (1H, t, J 10.0), 6.30 (1H, d, J 8.5), 6.95 (2H, s), 6.99 (2H, s), 7.04 (2H, s), 7.09 (2H, s), 7.16 (2H, s).

### Example 3

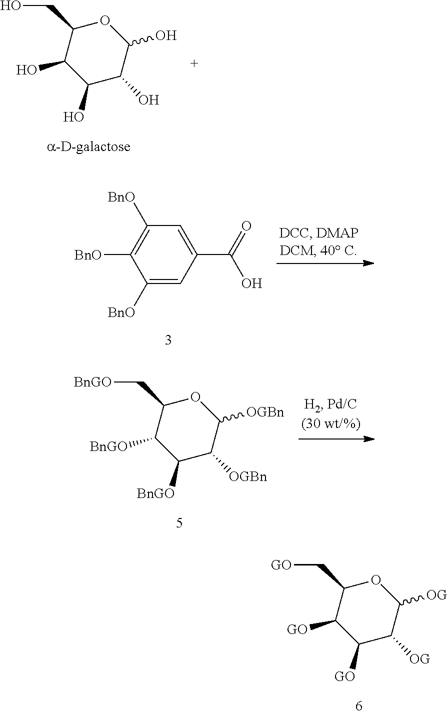

Compound 6. Compound 6 was prepared using the following synthesis:

Step 1: Starting from Compound 3 (7.3 eq) and α-D-galactose (1.0 eq) following procedure General Synthesis B to form Compound 5. Compound 5 was stirred for 16 hours at 60° C. under H₂ atmosphere (10 atm). The reaction was filtrated and the cake was washed with MeOH (10 V). The reactant was concentrated and purified by Prep-HPLC and identified as Compound 6 with an 81% yield.

### Example 4

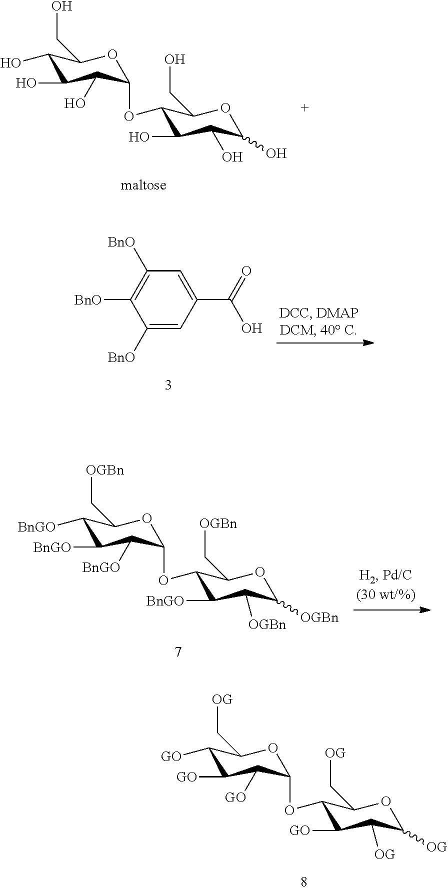

Compound 8. Compound 8 was prepared with the following synthesis:

Step 1: Starting from Compound 3 (7.3 eq) and maltose (1.0 eq) following procedure General Synthesis B to form Compound 7. Compound 7 was stirred for 16 hours at 60° C. under H₂ atmosphere (10 atm). The reaction was filtrated and the cake was washed with MeOH (10 V). The reactant was concentrated and purified by Prep-HPLC and identified as Compound 8.

### Example 5

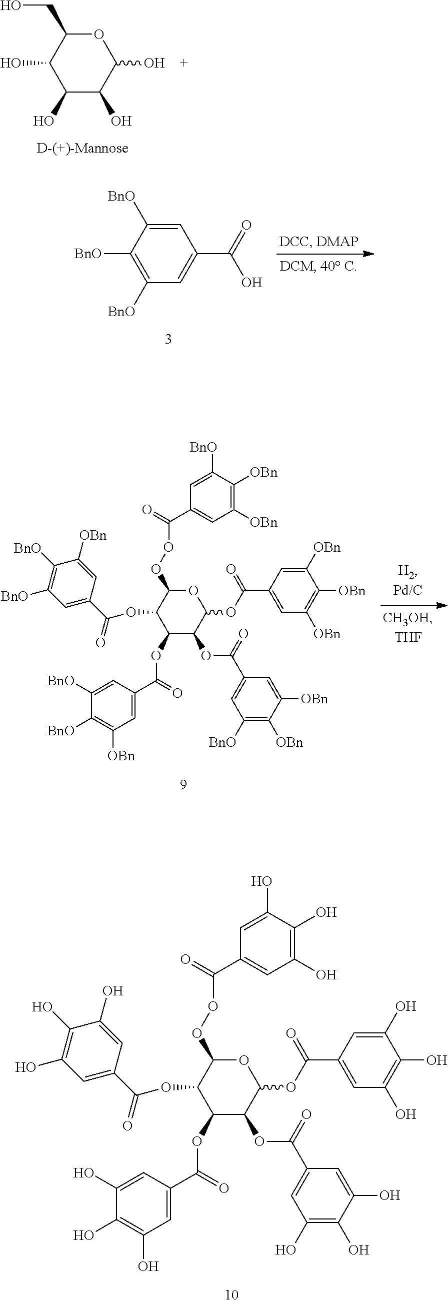

Compound 10. Compound 10 was prepared with the following synthesis:

To a stirred mixture of D-(+)-Mannose (3 g, 16.65 mmol, 1.0 equiv) and Compound 3 (55.01 g, 0.12 mol, 7.5 equiv) in DCM (1000.00 mL) were added DMAP (17.29 g, 0.14 mmol, 8.5 equiv) and DCC (30.92 g, 0.15 mmol, 9 equiv) at room temperature under nitrogen atmosphere. The resulting mixture was stirred for 16 h at 40° C. under nitrogen atmosphere. The resulting mixture was concentrated under reduced pressure. The residue was purified by silica gel column chromatography, eluted with PE/DCM (1:9) to afford Compound 9 as a white foam. To a stirred mixture of D-(+)-Mannose (3 g, 16.65 mmol, 1.0 equiv) and 3,4,5-tris (benzyloxy) benzoic acid (55.01 g, 0.12 mol, 7.5 equiv) in DCM (1000.00 mL) were added DMAP (17.29 g, 0.14 mmol, 8.5 equiv) and DCC (30.92 g, 0.15 mmol, 9 equiv) at room temperature under nitrogen atmosphere. The resulting mixture was stirred for 16 h at 40° C. under nitrogen atmosphere. The resulting mixture was concentrated under reduced pressure. The residue was purified by silica gel column chromatography, eluted with PE/DCM (1:9) to afford Compound 9 (24 g, 62.87%) as a white foam.

To a stirred mixture of Compound 9 in THF (200 mL) was added Pd/C (2.4 g) and CH₃OH (40 mL) at room temperature under nitrogen atmosphere. The resulting mixture was stirred for 16 h at 60° C. under hydrogen (10 atm) atmosphere. The mixture was allowed to cool down to room temperature. The resulting mixture was filtered, the filter cake was washed with MeOH (2×100 mL). The filtrate was concentrated under reduced pressure. The residue was purified by silica gel column chromatography, eluted with PE/THF (1:1) to afford Compound 10 (7.0880 g, 71.98%) as a white solid.

LCMS (ES, m/z): [M-H]⁻=939 ¹HNMR: (400 MHz, CD₃COCD₃, ppm) δ 8.22 (s, 7H), 7.34 (s, 2H), 7.27 (d, J=14.6 Hz, 4H), 7.18 (d, J=8.6 Hz, 4H), 7.15-7.05 (m, 6H), 6.99 (d, J=8.6 Hz, 4H), 6.53-6.48 (m, 2H), 6.04-5.86 (m, 3H), 5.81 (ddd, J=9.9, 3.6, 2.3 Hz, 3H), 4.73-4.64 (m, 1H), 4.60-4.44 (m, 5H).

### Example 6

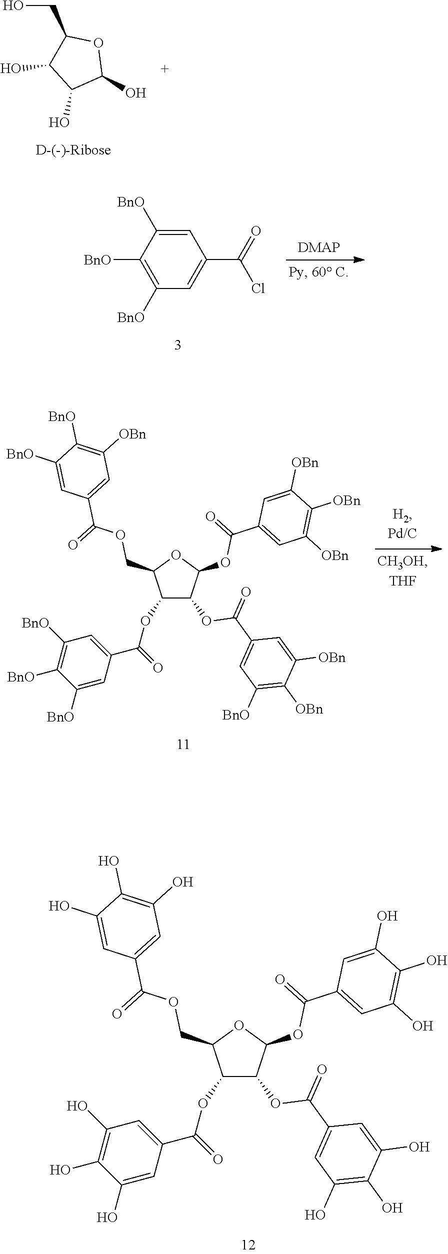 [SMILES: OC[C@H]1O[C@@H](O)[C@H](O)[C@@H]1O ; O=C(Cl)c1cc(OCc2ccccc2)c(OCc2ccccc2)c(OCc2ccccc2)c1 ; O=C(OC[C@H]1O[C@@H](OC(=O)c2cc(OCc3ccccc3)c(OCc3ccccc3)c(OCc3ccccc3)c2)[C@H](OC(=O)c2cc(OCc3ccccc3)c(OCc3ccccc3)c(OCc3ccccc3)c2)[C@@H]1OC(=O)c1cc(OCc2ccccc2)c(OCc2ccccc2)c(OCc2ccccc2)c1)c1cc(OCc2ccccc2)c(OCc2ccccc2)c(OCc2ccccc2)c1 ; O=C(OC[C@H]1O[C@@H](OC(=O)c2cc(O)c(O)c(O)c2)[C@H](OC(=O)c2cc(O)c(O)c(O)c2)[C@@H]1OC(=O)c1cc(O)c(O)c(O)c1)c1cc(O)c(O)c(O)c1]

Compound 12. Compound 12 was prepared with the following synthesis:

To a stirred mixture of D-(−)-Ribose (5 g, 33.30 mmol, 1.0 equiv) and Compound 3 (122.28 g, 266.43 mmol, 8 equiv) in Pyridine (500 mL) were added DMAP (0.41 g, 3.33 mmol, 0.1 equiv) at room temperature under nitrogen atmosphere. The resulting mixture was stirred for 48 h at 60° C. under nitrogen atmosphere. The product was precipitated by the addition of methanol. The residue was purified by silica gel column chromatography, eluted with PE/DCM (1:9) to afford Compound 11 (22 g, 35.90%) as a white foam.

To a stirred mixture of Compound 11 (22 g, 11.95 mmol, 1.0 equiv) in THF (190 mL) were added Pd/C (2.2 g) and CH₃OH (30 mL) at room temperature under nitrogen atmosphere. The resulting mixture was stirred for 16 h at 60° C. under nitrogen (10 atm) atmosphere. The mixture was allowed to cool down to room temperature. The resulting mixture was filtered, the filter cake was washed with MeOH (2×100 mL). The filtrate was concentrated under reduced pressure. The residue was purified by silica gel column chromatography, eluted with PE/THF (1:1) to afford Compound 12 (7.0681 g, 77.93%) as a white solid.

LCMS: (ES, m/z): [M-H]⁻=757 ¹HNMR: (400 MHZ, CD₃COCD₃, ppm) δ 8.16 (s, 1H), 7.21 (d, J=11.8 Hz, 4H), 7.10 (d, J=10.6 Hz, 4H), 6.43 (d, J=6.7 Hz, 1H), 6.14 (t, J=3.2 Hz, 1H), 5.57-5.49 (m, 2H), 4.34 (dd, J=11.6, 4.4 Hz, 1H), 4.23 (dd, J=11.7, 8.3 Hz, 1H).

### Example 7

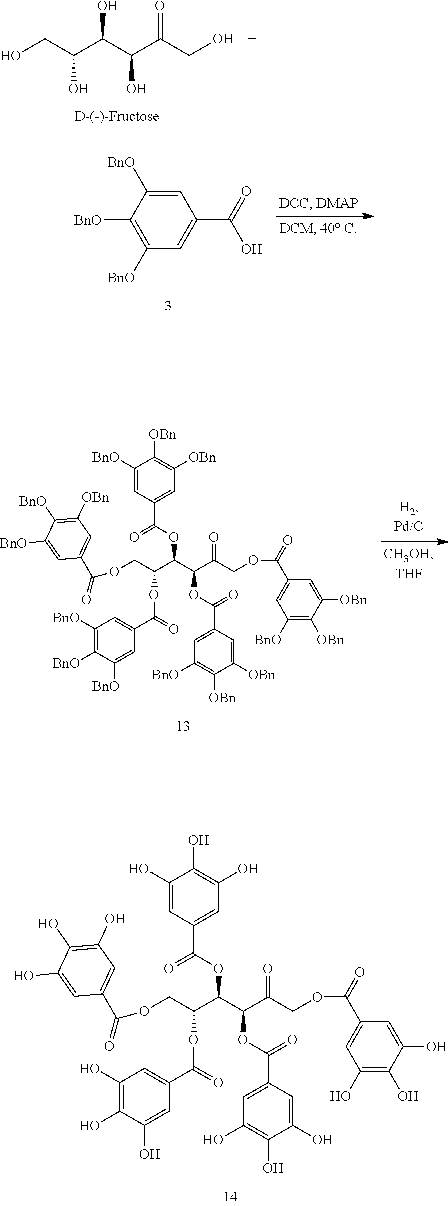

Compound 14. Compound 14 was prepared with the following synthesis:

To a stirred mixture of D-(−)-Fructose (70 g, 388.55 mmol, 1.0 equiv) and Compound 3 (1026.93 g, 2331.31 mmol, 6.0 equiv) in DCM (10 L) were added DCC (577.23 g, 2797.57 mmol, 7.2 equiv) and DMAP (379.76 g, 3108.41 mmol, 8 equiv) at room temperature under nitrogen atmosphere. The resulting mixture was stirred for 16 h at 40° C. under nitrogen atmosphere. The product was precipitated by the addition of MeOH. The residue was purified by silica gel column chromatography, eluted with PE/DCM (1:9) to afford Compound 13 (19 g, 2.13%) as a white foam.

To a stirred mixture of Compound 13 (19 g, 8.28 mmol, 1.0 equiv) in THF (200 mL) were added Pd/C (1.9 g, 17.85 mmol, 2.15 equiv) and CH₃OH (30 mL) at room temperature under nitrogen atmosphere. The resulting mixture was stirred for 16 h at 60° C. under hydrogen (10 atm) atmosphere. The mixture was allowed to cool down to room temperature. The resulting mixture was filtered, the filter cake was washed with MeOH (2×100 mL). The filtrate was concentrated under reduced pressure. The residue was purified by silica gel column chromatography, eluted with PE/THF (1:1) to afford Compound 16 (5.1248 g, 65.74%) as a white solid.

LCMS: (ES, m/z): [M-H]⁻=939 ¹HNMR: (400 MHZ, CD₃COCD₃, ppm) δ 8.21 (s, 1H), 7.25 (s, 2H), 7.23-7.14 (m, 6H), 7.13 (s, 2H), 6.46 (d, J=3.7 Hz, 1H), 5.66 (dd, J=6.1, 3.7 Hz, 1H), 5.02 (d, J=11.8 Hz, 1H), 4.97 (ddd, J=7.2, 6.1, 3.1 Hz, 1H), 4.88-4.76 (m, 2H), 4.50 (dd, J=12.1, 7.2 Hz, 1H).

### Example 8

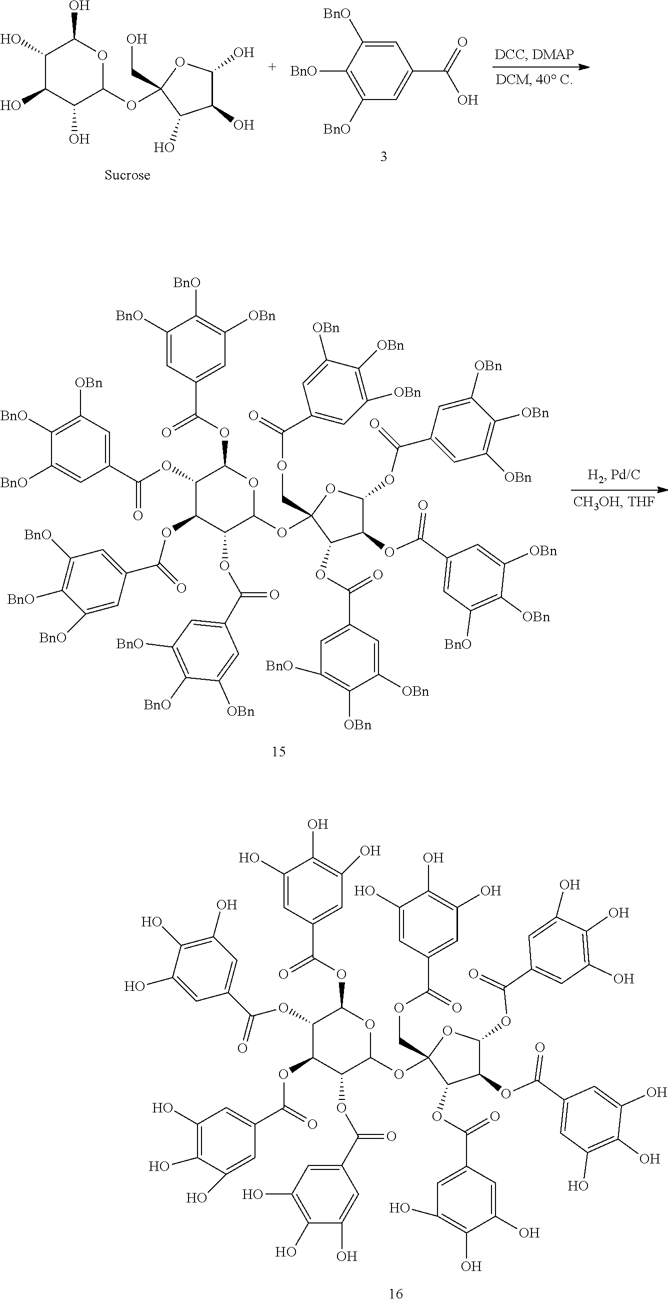

Compound 16. Compound 16 was prepared with the following synthesis:

To a stirred mixture of Sucrose (4 g, 11.68 mmol, 1.0 equiv) and Compound 3 (82.36 g, 186.97 mmol, 16 equiv) in DCM (1 L) were added DCC (34.72 g, 168.278 mmol, 14.4 equiv) and DMAP (22.84 g, 186.97 mmol, 16 equiv) dropwise in portions at room temperature under nitrogen atmosphere. The resulting mixture was stirred for 16 h at 40° C. under nitrogen atmosphere. The resulting mixture was concentrated under reduced pressure. The product was precipitated by the addition of MeOH. The residue was purified by silica gel column chromatography, eluted with PE/DCM (1:9) to afford Compound 15 (20 g, 45.98%) as a white foam.

To a stirred mixture of Compound 15 (20 g, 5.37 mmol, 1.0 equiv) in THF (200 mL) were added Pd/C (2 g) and CH₃OH (40 mL) at room temperature under nitrogen atmosphere. The resulting mixture was stirred for 16 h at 60° C. under hydrogen (10 atm) atmosphere. The mixture was allowed to cool down to room temperature. The resulting mixture was filtered, the filter cake was washed with MeOH (2×100 mL). The filtrate was concentrated under reduced pressure. The residue was purified by silica gel column chromatography, eluted with PE/THF (1:1) to afford Compound 16 (7.0564 g, 84.23%) as a light grey solid.

LCMS: (ES, m/z): [M-H]⁻=1557 ¹HNMR: (400 MHZ, CD₃COCD₃, ppm) δ 8.21 (s, 6H), 7.41 (s, 2H), 7.27 (s, 2H), 7.19 (d, J=7.4 Hz, 4H), 7.11 (d, J=12.1 Hz, 4H), 7.05 (s, 2H), 6.98 (s, 2H), 6.21-6.12 (m, 2H), 6.06 (d, J=7.9 Hz, 1H), 6.03-5.95 (m, 1H), 5.81 (t, J=10.0 Hz, 1H), 5.45 (dd, J=10.5, 3.5 Hz, 1H), 4.91-4.77 (m, 2H), 4.77-4.65 (m, 3H), 4.60 (dd, J=12.8, 2.7 Hz, 1H), 4.52-4.40 (m, 2H).

### Example 9

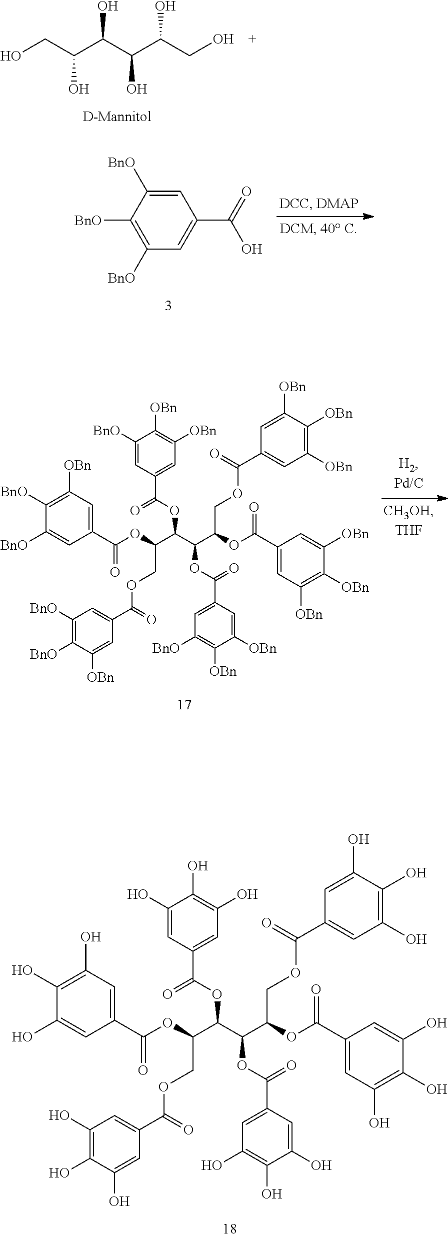

Compound 18. Compound 18 was prepared with the following synthesis:

To a stirred mixture of D-Mannitol (4 g, 21.95 mmol, 1.0 equiv) and Compound 3 (87.05 g, 197.61 mmol, 9 equiv) in DCM (1 L) were added DCC (48.93 g, 237.13 mmol, 10.8 equiv) and DMAP (27.36 g, 223.96 mmol, 10.2 equiv) at room temperature under nitrogen atmosphere. The resulting mixture was stirred for 16 h at 40° C. under nitrogen atmosphere. The product was precipitated by the addition of MeOH. The residue was purified by silica gel column chromatography, eluted with PE/DCM (1:9) to afford Compound 17 (24 g, 40.23%) as a white foam.

To a stirred mixture of Compound 17 (24 g, 8.83 mmol, 1.0 equiv) in THF (200 mL) were added Pd/C (2.4 g, 22.55 mmol, 2.55 equiv) and MeOH (40 mL) at room temperature under nitrogen atmosphere. The resulting mixture was stirred for 16 h at 60° C. under hydrogen (10 atm) atmosphere. The mixture was allowed to cool down to room temperature. The resulting mixture was filtered, the filter cake was washed with MeOH (2×100 mL). The filtrate was concentrated under reduced pressure. The residue was purified by silica gel column chromatography, eluted with PE/THF (1:1) to afford Compound 18 (6.0744 g, 62.81%) as a light grey solid.

LCMS: (ES, m/z): [M-H]⁻=1093 ¹HNMR: (400 MHZ, CD₃COCD₃, ppm) δ 8.18 (s, 1H), 7.23 (s, 2H), 7.13 (s, 2H), 7.06 (s, 2H), 6.08-6.00 (m, 1H), 5.77 (td, J=7.3, 2.3 Hz, 1H), 4.71 (dd, J=12.3, 2.5 Hz, 1H), 4.55 (dd, J=12.2, 7.8 Hz, 1H).

### Example 10

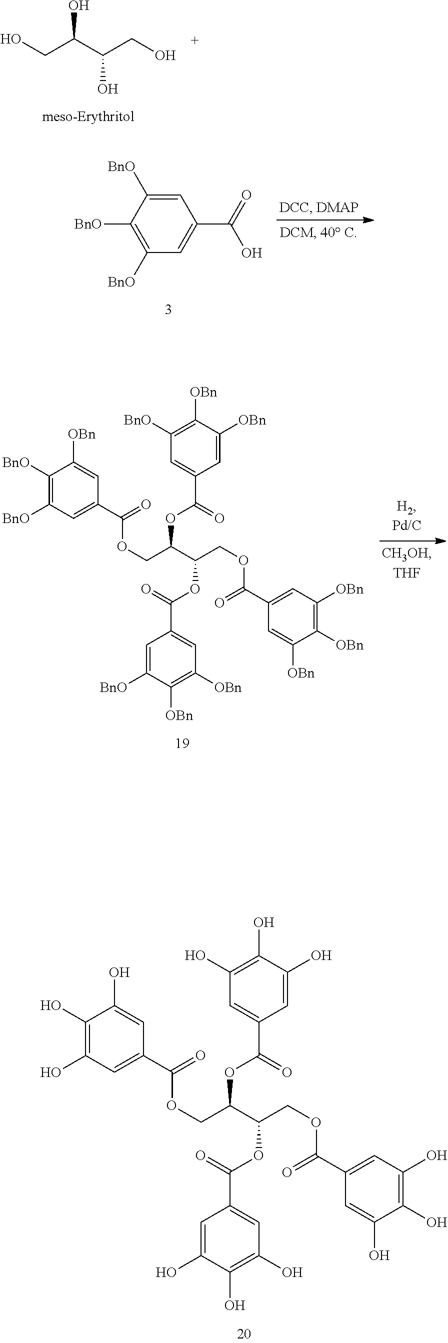 [SMILES: OC[C@@H](O)[C@@H](O)CO ; O=C(O)c1cc(OCc2ccccc2)c(OCc2ccccc2)c(OCc2ccccc2)c1 ; O=C(OC[C@H](OC(=O)c1cc(OCc2ccccc2)c(OCc2ccccc2)c(OCc2ccccc2)c1)[C@@H](COC(=O)c1cc(OCc2ccccc2)c(OCc2ccccc2)c(OCc2ccccc2)c1)OC(=O)c1cc(OCc2ccccc2)c(OCc2ccccc2)c(OCc2ccccc2)c1)c1cc(OCc2ccccc2)c(OCc2ccccc2)c(OCc2ccccc2)c1 ; O=C(OC[C@H](OC(=O)c1cc(O)c(O)c(O)c1)[C@@H](COC(=O)c1cc(O)c(O)c(O)c1)OC(=O)c1cc(O)c(O)c(O)c1)c1cc(O)c(O)c(O)c1]

Compound 20. Compound 20 was prepared with the following synthesis:

To a stirred mixture of meso-Erythritol (4 g, 32.75 mmol, 1.0 equiv) and Compound 3 (86.57 g, 196.53 mmol, 6 equiv) in DCM (1 L) were added DCC (48.66 g, 235.83 mmol, 7.2 equiv) and DMAP (32.01 g, 262.04 mmol, 8.0 equiv) at room temperature under nitrogen atmosphere. The resulting mixture was stirred for 16 h at 40° C. under nitrogen atmosphere. The resulting mixture was concentrated under reduced pressure. The product was precipitated by the addition of MeOH. The residue was purified by silica gel column chromatography, eluted with PE/DCM (1:9) to afford Compound 19 (32 g, 53.91%) as a white foam.

To a stirred mixture of Compound 19 (32 g, 17.66 mmol, 1.0 equiv) in THF (300 mL) were added Pd/C (3.2 g, 30.07 mmol, 1.70 equiv) and MeOH (50 mL) at room temperature under nitrogen atmosphere. The resulting mixture was stirred for 16 h at 60° C. under hydrogen (10 atm) atmosphere. The mixture was allowed to cool down to room temperature. The resulting mixture was filtered, the filter cake was washed with MeOH (2×100 mL). The filtrate was concentrated under reduced pressure. The residue was purified by silica gel column chromatography, eluted with PE/THF (1:1) to afford Compound 20 (7.0498 g, 54.64%) as a white solid.

LCMS: (ES, m/z): [M-H]⁻=729 ¹HNMR: (400 MHZ, CD₃COCD₃, ppm) δ 8.02 (s, 1H), 7.16 (d, J=16.1 Hz, 20H), 5.85-5.79 (m, 5H), 4.81 (dd, J=12.1, 2.5 Hz, 5H), 4.65-4.56 (m, 5H).

### Example 11

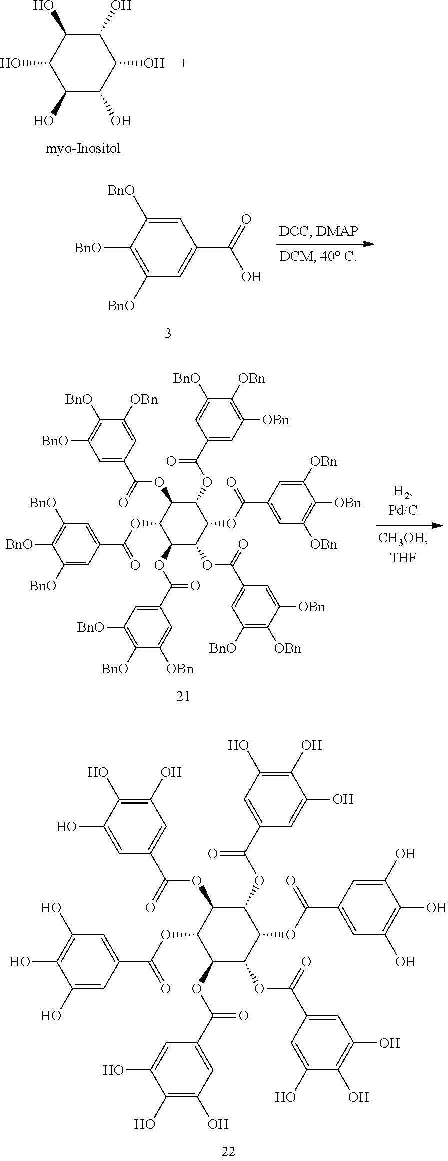

Compound 22. Compound 22 was prepared with the following synthesis:

To a stirred mixture of myo-Inositol (4 g, 22.20 mmol, 1.0 equiv) and Compound 3 (88.02 g, 199.82 mmol, 9 equiv) in DCM (1 L) were added DCC (49.48 g, 239.79 mmol, 10.8 equiv) and DMAP (27.67 g, 226.47 mmol, 10.2 equiv) at room temperature under nitrogen atmosphere. The resulting mixture was stirred for 16 h at 40° C. under nitrogen atmosphere. The resulting mixture was concentrated under reduced pressure. The product was precipitated by the addition of MeOH. The residue was purified by silica gel column chromatography, eluted with PE/DCM (1:9) to afford Compound 21 (17 g, 28.20%) as a white foam.

To a stirred mixture of Compound 21 (17 g, 6.26 mmol, 1.0 equiv) in THF (200 mL) were added Pd/C (1.7 g) and MeOH (30 mL) at room temperature under nitrogen atmosphere. The resulting mixture was stirred for 16 h at 60° C. under hydrogen (10 atm) atmosphere. The mixture was allowed to cool down to room temperature. The resulting mixture was filtered, the filter cake was washed with MeOH (2×100 mL). The filtrate was concentrated under reduced pressure. The residue was purified by silica gel column chromatography, eluted with PE/THF (1:1) to afford Compound 22 (6.0576 g, 88.53%) as a light grey solid.

LCMS: (ES, m/z): [M-H]⁻=1091 ¹HNMR: (400 MHZ, CD₃COCD₃, ppm) δ 8.15 (s, 1H), 7.26 (s, 2H), 7.04-6.96 (m, 10H), 6.30-6.18 (m, 3H), 5.90 (dd, J=10.2, 3.0 Hz, 2H).

### Example 12

This example illustrates a Phase 1 study of embodiments of the disclosure.

The aorta, as the largest artery in the human body, plays a pivotal role in cardiovascular physiology by conducting oxygenated blood from the heart to various tissues via systemic circulation. Structurally, the aorta is composed of three distinct layers: the intima, the innermost layer providing a smooth endothelial lining; the media, a middle layer constituted of concentric sheets of elastic fibers, collagen, and smooth muscle cells that confer the vessel's strength and elasticity; and the adventitia, an outer layer comprised of connective tissue that supports and protects the aortic wall. This tri-layered architecture is essential for the aorta's function, allowing it to withstand and modulate the pulsatile pressure generated by the heart, ensuring efficient blood flow. The aortic wall's microstructure, particularly the configuration and interaction of its elastic and collagenous components, plays an important role in its biomechanical properties, including compliance and resilience, which are indispensable for maintaining hemodynamic stability and adapting to physiological demands. Disruptions or anomalies in the aortic wall's microstructure can lead to significant cardiovascular diseases, such as aneurysms and dissections.

Aneurysms grow over a period of years and pose great risks to health. Aneurysms have the potential to dissect or rupture, causing massive bleeding, stroke, and hemorrhagic shock, which can be fatal in more than 80% of cases. AAAs are a serious health concern, specifically for the aging population, being among the top ten causes of death for patients older than 50. The estimated incidence for abdominal aortic aneurysm is about 50 in every 100,000 persons per year. Approximately 50,000 operations are performed each year in the U.S. for AAAs alone. In children, AAAs can result from blunt abdominal injury or from Marfan's syndrome, a defect in elastic fiber formation in walls of major arteries, such as the aorta.

Current methods of treatment for diagnosed aneurysms are limited to invasive surgical techniques. After initial diagnosis of a small aneurysm, the most common medical approach is to follow up the development of the aneurysm and after reaching a pre-determined size (for example, about 5 cm in diameter), surgical treatment is applied. Current surgical treatments are limited to either an endovascular stent graft repair or optionally complete replacement of the diseased vessel with a vascular graft. While such surgical treatments can save lives and improve quality of life for those suffering aneurysms, dangers beyond those of the surgery itself still exist for the patient due to possible post-surgery complications (for example, neurological injuries, bleeding, or stroke) as well as device-related complications (for example, thrombosis, leakage, or failure). Moreover, depending upon the location of the aneurysm, the danger of an invasive surgical procedure may outweigh the possible benefits of the procedure, for instance in the case of an aneurysm deep in the brain, leaving the sufferer with very little in the way of treatment options. Moreover, surgical treatments may not always provide a permanent solution, as vascular grafts can loosen and dislodge should the aneurysm progress following the corrective surgery. For some patients, the particular nature of the aneurysm or the condition of the patient makes the patient unsuitable for graft repair.

β-PGG, similar to other tannins, has been recognized for use in a wide variety of application and exerts its biological effects primarily through its capacity to form complexes with proteins. This action is mediated by hydrophobic interactions and hydrogen bonding, which lead to protein precipitation. Notably, when β-PGG is introduced to cardiovascular tissues, it uniquely engages with key structural proteins, elastin, and collagen. These regions within the extracellular matrix (ECM) are notably vulnerable to degradation by proteases, a process known as elastolysis, which can compromise the integrity of the tissue. Although the specific molecular interactions are complex and not fully elucidated, it is proposed-without commitment to a particular theory—that β-PGG's ability to bind to aortic elastin could reinforce the elastin's structural integrity. This reinforcement could potentially restrict the dilatation of the aorta that leads to aneurysm formation. However, prior to this work, it was uncertain if other compounds, as those described herein, could also bind to aortic elastin and similarly prevent aneurysmal growth.

In this study, compounds of this disclosure were added to abdominal aortas in an opening angle experiment to determine how they would affect the aorta in vitro. Thus, to understand the ability of the compounds of this disclosure to bind to aortic elastin and determine their ability to affect the aorta and aneurysms.

### Materials and Methods—Scan of Analogs

Twelve porcine abdominal aortas were ordered and received. The infra-renal sections of these aortas were dissected, cleaned, rinsed, and then cut into rings measuring 3 mm in width. From each artery, 10-11 rings were obtained, resulting in a total of 122 rings. These rings were mixed up before testing to randomize them based on their location in the artery. They were then stored in 500 ml of saline. Analog solutions were prepared at the same molar concentration, but they were not normalized for gallic acid content as indicated in Table 1. These solutions of analogs were made using a solution containing about 26% iopamidol, a radioopaque contrast agent. The solutions had a pH of 3.7, and the analogs were stable for at least 2 weeks. All solutions were prepared one day prior to their use. On the day of use, the solution's pH was adjusted with a 4.2% sodium bicarbonate solution at a ratio of 15:1 (20 ml of Analog solution to 1.33 ml of bicarbonate). For each solution, 6 aortic rings were placed in a pre-labeled 50 ml conical tube containing 20 ml of the solution, resulting in a volume to tissue ratio of approximately 3.3 ml of solution per ring. The tubes were then incubated at room temperature for 3 hours, with rotation every 30-40 minutes.

After the treatment, while still in the tube, the solution was decanted, and the rings were rinsed three times in PBS and then suspended in PBS. The rings were placed in a 6-well plate, with 2 rings per well and 3 ml of PBS in each well. Each ring was opened with a single cut using fine scissors and laid back in the well. The rings were allowed 15 minutes to recoil in the PBS at room temperature, with gentle shaking every 5 minutes.

Photographs of the rings were taken, and their opening angles were measured using Image J software. The data were then plotted and statistical analyses were performed for comparison.

TABLE 1

| | | | | Starting | | |
|---|---|---|---|---|---|---|
| | | | Molar | Conc. | Final conc. | |
| | | | (Final | (Stock | after pH | |
| | | | molarity) | Soln) | adjustment | Sample |
| | Gallate | | Conc. | Conc. | Conc. | size |
| Group | Derivative | MW | (mM) | (mg/mL) | (mg/mL) | (rings) |
| D | α-PGG | 940 | 2.49 | 2.50 | 2.34 | 6 |
| E | β-PGG | 940 | 2.49 | 2.50 | 2.34 | 6 |
| J | PG-mannose | 940 | 2.49 | 2.50 | 2.34 | 6 |
| F | PG-galactose | 940 | 2.49 | 2.50 | 2.34 | 6 |
| L | TG-ribose | 758 | 2.49 | 2.01 | 1.89 | 6 |
| M | PG-fructose | 940 | 2.49 | 2.50 | 2.34 | 6 |
| N | OG-sucrose | 1530 | 2.49 | 4.06 | 3.81 | 6 |
| G | OG-maltose | 1559 | 2.49 | 4.14 | 3.88 | 6 |
| P | HG-mannitol | 1094 | 2.49 | 2.90 | 2.72 | 6 |
| S | HG-inositol | 1092 | 2.49 | 2.90 | 2.72 | 6 |

Controls

| B | Propyl gallate | 212 | 2.49 | 0.56 | 0.53 | 6 |
|---|---|---|---|---|---|---|
| B5 | 5xPropyl gallate | 212 | 12.45 | 2.82 | 2.64 | 6 |
| T | Glucose | 180 | 2.49 | 0.48 | 0.45 | 6 |
| U | Gallic acid | 170 | 2.49 | 0.45 | 0.42 | 6 |
| A | 26% Iopamidol | N/A | | | | 6 |
| I | Glutaraldehyde | N/A | 0.5% in PBS, pH 7.4 | | | 6 |
| X | PBS | N/A | N/A | | | 6 |

### Results

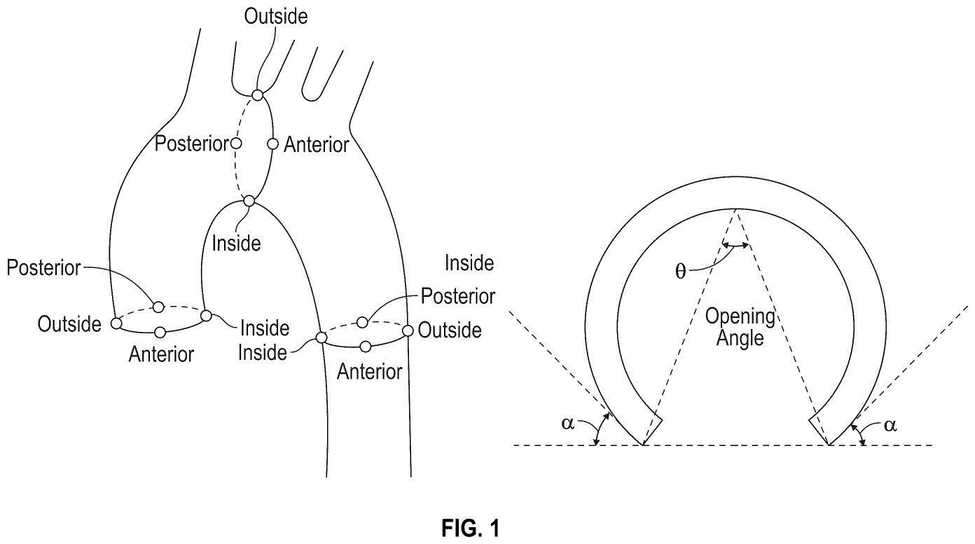

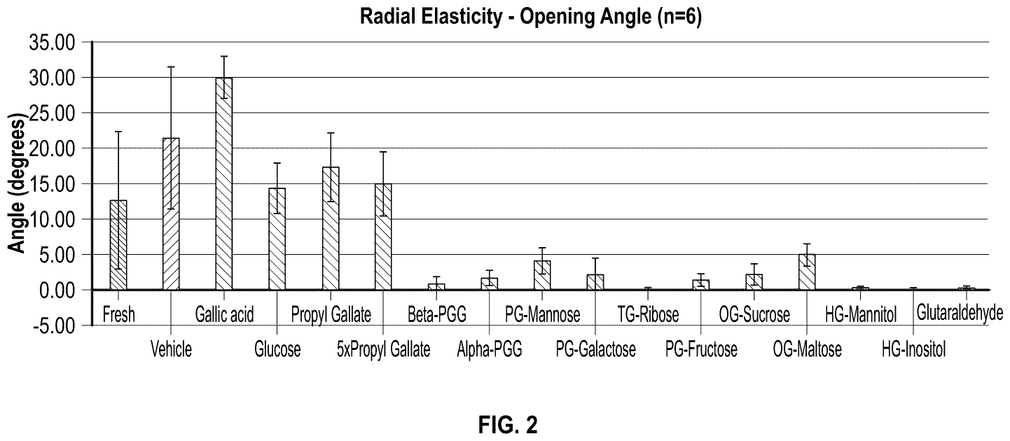

The data on opening angles were categorized into two main groups: “controls” and “analogs.” FIG. 1 shows a sketch of an aorta and a thin ring cut from the aorta that is cut again radially. The ring opens up into a sector. Neither the ring nor the sector is exactly circular. For geometric description, two radii were drawn from the midpoint of the arc of the inner wall of the vessel to the outer tips of the sector. The angles between the tangents of the sector at the tips and the line joining the tips are designated a and a!. The mean value (a+a′) 2 is measured and is called the mean tangential angle. If the section is exactly circular, then 0 (a+a′) 2. The opening angle is, therefore, seen as a result of release of stress in the connective tissue rather than one of smooth muscle contraction. The control group consisted of six subgroups: PBS, 26% Iopamidol, Gallic acid, Glucose, Propyl-gallate, and Propyl gallate 5×, which are the first six presented on the left in FIG. 2. The mean opening angles for these groups ranged from 12 to 30 degrees. Each control group displayed significant variability, with 2-3 observations showing high opening angles (30-60 degrees) and 2-3 observations showing very low opening angles (0-10 degrees). However, there was no significant statistical difference observed among these six control groups, as determined by ANOVA (p<0.05).

The “analogs” group encompassed all the analog compounds from β-PGG to HG-inositol, with Glutaraldehyde serving as the positive control. This group generally showed very low mean opening angles, although some variability was noted, including 3-4 instances of zero-degree angles and 2-3 instances of angles ranging from 5 to 13 degrees. Compared to the PBS subgroup, all analogs demonstrated statistically significant differences, except for OG-maltose. Similarly, when compared to the 26% Iopamidol control subgroup, all analogs showed statistically significant differences.

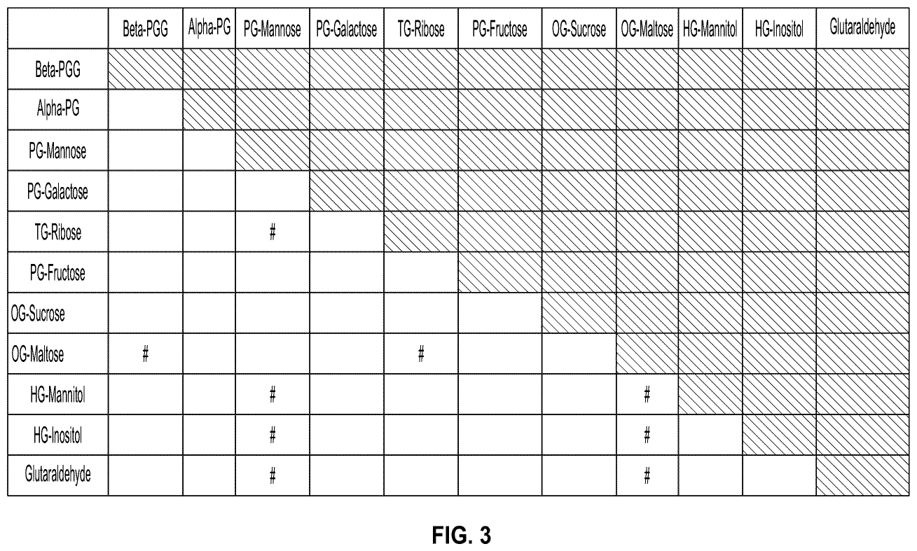

A statistical analysis of the analogs revealed two distinct sub-groups based on their mean opening angles as depicted in FIG. 3. One sub-group consisted of analogs with very low mean opening angles, less than 1 degree, which included β-PGG, TG-ribose, HG-mannitol, HG-inositol, and Glutaraldehyde. The second sub-group had mean opening angles ranging from 1 to 4.8 degrees and comprised the remaining analogs. The comparison showed that alpha and beta PGG were not statistically different. In contrast, PG-mannose and OG-maltose were statistically higher than beta-PGG, TG-ribose, HG-mannitol, HG-inositol and Glutaraldehyde.

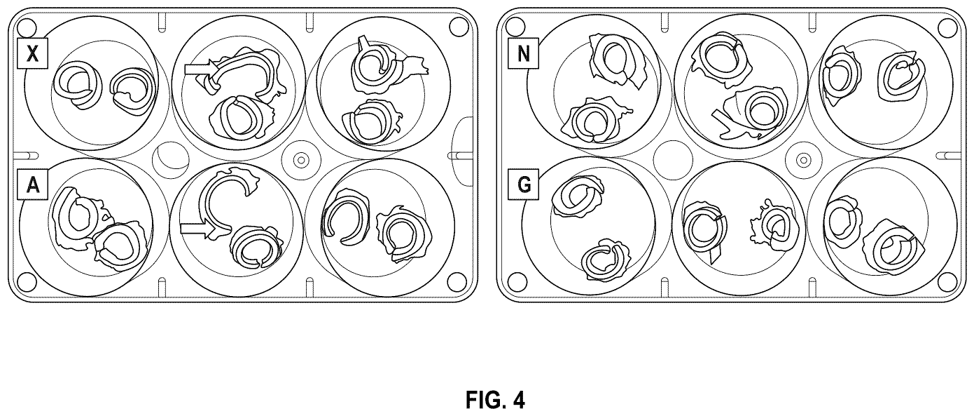

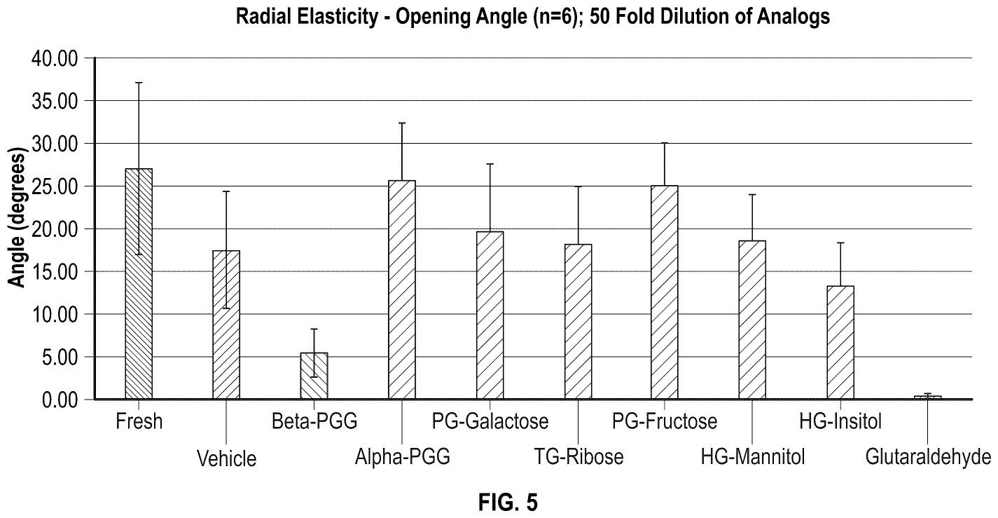

In the context of control samples, the PBS (Fresh) group (X) had one instance where a ring opened to a large degree, to 60 degrees, as highlighted by an arrow in FIG. 4. The rest of the rings in this group showed minimal opening. Similarly, in the 26% Iopamidol (Vehicle) group (A), one ring opened to 70 degrees (indicated by an arrow), another to 20 degrees, while the others had very low opening angles. Other control samples showed less variability. This variability was substantially reduced in the groups treated with analogs, as illustrated in FIG. 5 for the samples N (OG sucrose) and G (OG-maltose).

The findings indicated that all tested analogs were effective in reducing the opening angles of porcine abdominal aortas, aligning with the effect observed with β-PGG. Notably, three analogs-TG-ribose, HG-mannitol, and HG-inositol-showed statistically significant lower opening angles, suggesting potentially stronger interactions with elastin. Thus, these in vitro findings suggest that the compounds of the disclosure can be used in aortopathy therapeutics.

### Example 13

In the analysis of aneurysms, determining the stress-free state of the tissue is studied, yet often, detailed information on this state is lacking. The presence of residual stresses in the arterial wall's circumferential direction can be illustrated by cutting a ring of the artery along its axial direction. The opening angle, a key biomechanical parameter, measures the residual strain in blood vessels or tubular organs when they are cut radially and unfold into a sector shape. This measurement may be instrumental in revealing the arteries' behaviors and properties, particularly their capacity to withstand and evenly distribute mechanical stress under different physiological conditions. It can shed light on the vascular wall's structural integrity and functionality, including the impact of diseases like aneurysms. In healthy vessels, the opening angle's depiction of residual strain distribution is essential for maintaining optimal functioning and ability to meet the mechanical demands of blood circulation. Conversely, in disease states, alterations in the opening angle may signal changes in the vessel wall's structure and mechanics, potentially affecting aneurysm progression. The opening angle that results when the artery segment is allowed to open can be precisely measured optically, once the cut section stabilizes, providing a direct characterization of the circumferential residual strain. However, acquiring opening angle measurements from aneurysmal tissue poses challenges due to the diseased vessel wall's irregular shape. The analysis considers several factors: the change in wall composition, particularly the reduced elastin, which leads to increased viscoelastic behavior; the variability of residual strains across the wall's circumferential direction and its three layers; and the need to assess the residual stresses of these layers individually to determine if the intima is compressed while the media is stretched. Thus, research into opening angle measurements can be important for understanding aneurysms, as it aids in identifying vascular system areas under significant stress and prone to aneurysmal development.

To investigate the specific effect the analogs had on the opening angle, the analogs were prepared at a 50× diluted concentration, as specified in Table 2. The solutions had a pH of 3.7. All solutions were prepared one day prior to their use. On the day of the experiment, the solution's pH was adjusted using a 4.2% sodium bicarbonate solution at a ratio of 15:1 (30 ml of the Analog solution to 2 ml of bicarbonate).

TABLE 2

| | | | | Starting | Final | |
|---|---|---|---|---|---|---|
| | | | Molar | Conc. | conc. | |
| | | | (Final | (Stock | after pH | |
| | | | molarity) | Soln) | adjustment | Sample |
| | Gallate | | Conc. | Conc. | Conc. | size |
| Group | Derivative | MW | (mM) | (mg/mL) | (mg/mL) | (rings) |
| D | α-PGG | 940 | 0.05 | 0.05 | 0.0468 | 9 |
| E | β-PGG | 940 | 0.05 | 0.05 | 0.0468 | 9 |
| F | PG-galactose | 940 | 0.05 | 0.05 | 0.0468 | 9 |
| L | TG-ribose | 758 | 0.05 | 0.04 | 0.0378 | 9 |
| M | PG-fructose | 940 | 0.05 | 0.05 | 0.0468 | 9 |
| P | HG-mannitol | 1094 | 0.05 | 0.058 | 0.0544 | 9 |
| S | HG-inositol | 1092 | 0.05 | 0.058 | 0.0544 | 9 |

Controls

| A | 26% | N/A | | 9 |
|---|---|---|---|---|
| | Iopamidol | | | |
| I | Glutaraldehyde | N/A | 0.5% in PBS, pH 7.4 | 9 |
| X | PBS | N/A | PBS | 9 |

For this experiment, nine aortic rings were placed in 30 ml of a pre-prepared solution within a 50 ml conical tube. This setup resulted in a volume-to-tissue ratio of approximately 3.3 ml of solution for each ring. The tubes were then incubated at room temperature for 3 hours, with rotations occurring every 30-40 minutes. After the incubation period, while still in the tubes, the solution was poured off, and the rings were rinsed three times with PBS and then left suspended in PBS. The rings were then transferred to a 6-well plate, with 2 rings per well and 3-4 ml of PBS in each well. Each ring was opened with a single cut using fine scissors and laid flat in the well. They were allowed to recoil for 15 minutes in PBS at room temperature, with gentle shaking every 5 minutes. Photographs were taken, and the opening angle was measured using the Image J software.

In this phase of the study, the analogs were tested at a 50-fold dilution compared to their concentration in previous Example. The control groups included PBS (Fresh), 26% Iopamidol (Vehicle), β-PGG, and Glutaraldehyde (as a positive control), with the sample size increased to n=9 for each group. The six analogs showed results similar to the Fresh and Vehicle controls, indicating that the 50-fold dilution resulted in concentrations too low to detect any significant effects on elastic recoil. Statistical analysis showed no significant differences between the analogs at this dilution level and the PBS or 26% Iopamidol controls, with an ANOVA yielding a p-value of less than 0.05. However, β-PGG and Glutaraldehyde were found to be significantly different from all controls and analogs. These results are depicted in FIGS. 4 and 5.

Although the invention has been described with reference to embodiments and examples, it should be understood that numerous and various modifications can be made without departing from the spirit of the invention. Accordingly, the invention is limited only by the following claims.

It is understood that this disclosure, in many respects, is only illustrative of the numerous alternative device embodiments of the present invention. Changes may be made in the details, particularly in matters of shape, size, material and arrangement of various device components without exceeding the scope of the various embodiments of the invention. Those skilled in the art will appreciate that the exemplary embodiments and descriptions thereof are merely illustrative of the invention as a whole. While several principles of the invention are made clear in the exemplary embodiments described above, those skilled in the art will appreciate that modifications of the structure, arrangement, proportions, elements, materials and methods of use, may be utilized in the practice of the invention, and otherwise, which are particularly adapted to specific environments and operative requirements without departing from the scope of the invention. In addition, while certain features and elements have been described in connection with particular embodiments, those skilled in the art will appreciate that those features and elements can be combined with the other embodiments disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (for example, compositions and apparatuses including device and methods). For example, the term “comprising” will be understood to imply the inclusion of any stated elements or steps but not the exclusion of any other elements or steps.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of reducing or inhibiting growth of an aneurysm in a subject having an aneurysm, the method comprising:
administering a composition comprising at least one polygalloyl compound, or a salt thereof, a water-soluble solubilizer, and a buffer,
wherein the polygalloyl compound is not pentagalloyl β-glucose (β-PGG).

2. The method of claim 1, wherein the aneurysm is selected from an aortic aneurysm, a peripheral aneurysm, abdominal aortic aneurysm, or a neuro aneurysm.

3. The method of claim 2, wherein the aortic aneurysm is greater than 3 cm in diameter.

4. The method of claim 1, wherein the composition is administered by local delivery by a catheter device.

5. The method of claim 1, wherein the at least one polygalloyl compound is fully gallic acid substituted monosaccharides or disaccharides with the proviso that the one or more polygalloyl compound is not β-PGG.

6. The method of claim 1, wherein the polygalloyl compound is selected from the group consisting of polygalloyl galactose, polygalloyl xylose, polygalloyl ribose, polygalloyl arabinose, polygalloyl fructose, polygalloyl inositol, polygalloyl allose, polygalloyl talose, polygalloyl idose, polygalloyl gulose, polygalloyl erthrose, polygalloyl threose, polygalloyl sucrose, polygalloyl lactose, polygalloyl maltose, polygalloyl maltose, polygalloyl isomaltose, polygalloyl kojibiose, polygalloyl nigerose, polygalloyl laminaribiose, polygalloyl mannobiose, polygalloyl chitobiose, polygalloyl fucosyllactose, polygalloyl galactobiose, polygalloyl rutinose, polygalloyl neotrehalose, polygalloyl leucrose, polygalloyl trehalose, polygalloyl cellobiose, polygalloyl sorbitol, polygalloyl mannitol, polygalloyl maltitol, polygalloyl galactitol, polygalloyl fucitol, polygalloyl isomalt, polygalloyl xylitol, polygalloyl ribitol, polygalloyl ribitol, polygalloyl arabitol, polygalloyl erythritol, or combinations thereof.

7. The method of claim 6, wherein the at least one polygalloyl compound is a compound of Formula (III):

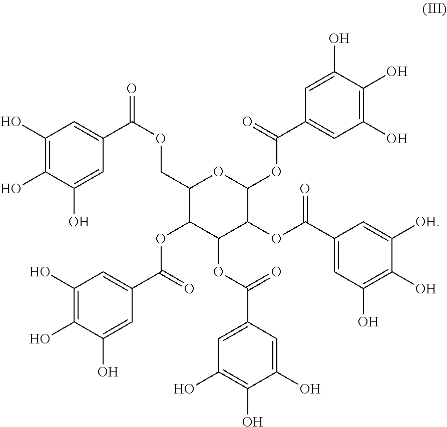 [SMILES: O=C(OCC1OC(OC(=O)c2cc(O)c(O)c(O)c2)C(OC(=O)c2cc(O)c(O)c(O)c2)C(OC(=O)c2cc(O)c(O)c(O)c2)C1OC(=O)c1cc(O)c(O)c(O)c1)c1cc(O)c(O)c(O)c1]

8. The method of claim 6, wherein the at least one polygalloyl compound is a compound of Formula (IV):

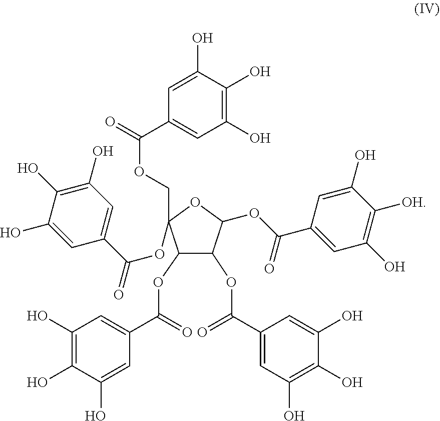 [SMILES: O=C(OCC1(OC(=O)c2cc(O)c(O)c(O)c2)OC(OC(=O)c2cc(O)c(O)c(O)c2)C(OC(=O)c2cc(O)c(O)c(O)c2)C1OC(=O)c1cc(O)c(O)c(O)c1)c1cc(O)c(O)c(O)c1]

9. The method of claim 6, wherein the at least one polygalloyl compound is a compound of Formula (V):

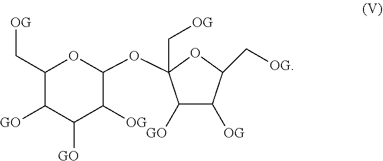

10. The method of claim 1, wherein the at least one polygalloyl compound is a compound of Formula (VI):

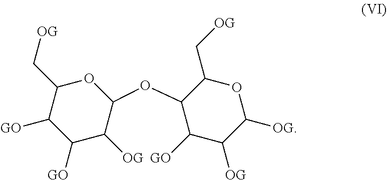

11. The method of claim 1, wherein the at least one polygalloyl compound is a compound of Formula (VII):

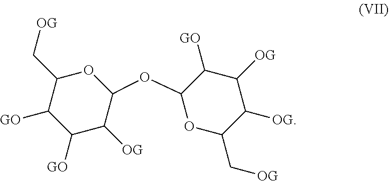

12. The method claim 1, wherein the at least one polygalloyl compound is a compound of Formula (VIII):

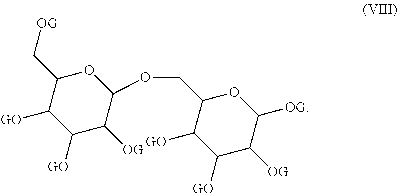

13. The method of claim 6, wherein the at least one polygalloyl compound is a compound of Formula (IX): 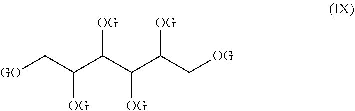
wherein, G is galloyl.

14. The method of claim 6, wherein the wherein the at least one polygalloyl compound is a compound of Formula (X): 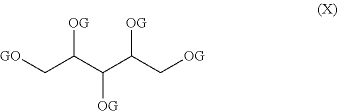
wherein, G is galloyl.

15. The method claim 6, wherein the at least one polygalloyl compound is a compound of Formula (XI): 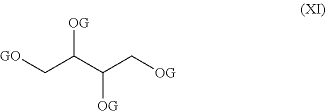
wherein, G is galloyl.

16. The method of claim 6, wherein the water-soluble solubilizer is a contrast agent.

17. The method of claim 16, wherein the contrast agent is a radiocontrast material from about 10 to about 30% (w/v) of the composition.

18. The method of claim 17, wherein the contrast agent is a radiocontrast material is a non-ionic radio-opaque contrast agent selected from the group consisting of iopamidol, iohexol, iodixanol, or a combination thereof.

19. A method for treating, preventing, ameliorating, or reducing an aortopathic condition or disease in a subject in need thereof, the method comprising:
administering a composition comprising a polygalloyl compound, or a salt thereof, a water-soluble solubilizer, and a buffer,
wherein the polygalloyl compound is not pentagalloyl β-glucose (β-PGG).

20. A method for blood vessel elastin stabilization in a subject in need thereof, the method comprising,
administering a composition comprising a polygalloyl compound, or a salt thereof, a water-soluble solubilizer, and a buffer,
wherein the polygalloyl compound is not pentagalloyl β-glucose (β-PGG).
